(19) United States
(12) Patent Application Publication
(10) Pub. No.: US 2024/0239747 A1
(43) Pub. Date: Jul. 18, 2024

(54) Next Generation 5-HT2AR Agonists

(71) Applicant: Huxley Health Inc., Concord (CA)

(72) Inventors: John Trant, Windsor (CA); Abd Al-Aziz A. Abu-Saleh, Windsor (CA); Muhammad Usman Mirza, Windsor (CA); Jonathan Simone, Windsor (CA)

(21) Appl. No.: 18/394,960

(22) Filed: Dec. 22, 2023

Related U.S. Application Data
(60) Provisional application No. 63/434,994, filed on Dec. 23, 2022.

(51) Int. Cl. C07D 209/16; C07D 405/12; C07D 409/12

(52) U.S. Cl. CPC C07D 209/16; C07D 405/12; C07D 409/12

(57) ABSTRACT

Disclosed are a variety of structures for agonists of 5-HT2AR some of which show promise for activity greater than psilocin. The agonists of the present disclosure are expected to demonstrate therapeutic benefits to treat mental disorders. In some embodiments, the invention may comprise the use of the compounds of the present invention to treat depression, and particularly drug resistant depression. Other conditions that may be treated include: anxiety disorders, including anxiety in advanced stage illness e.g. cancer as well as generalized anxiety disorder, depression including major depressive disorder, postpartum depression, cluster headaches, obsessive compulsive disorder, personality disorders including conduct disorder, drug disorders including: alcohol dependence, nicotine dependence, opioid dependence, cocaine dependence and other addictions including gambling disorder, eating disorder and body dysmorphic disorder, chronic pain, or chronic fatigue.

### CROSS REFERENCE TO RELATED APPLICATION

This application is related to U.S. provisional patent application Ser. No. 63/434,994 that was filed on 23 Dec. 2023 and which is hereby incorporated by reference.

### FIELD OF THE INVENTION

The invention relates to the next generation of tryptamine and phenethylamine 5-HT2AR agonists.

### BACKGROUND OF THE INVENTION

Psychiatric conditions contribute substantially to global disease burden. Treatment typically focuses on a combination of psychotherapy and pharmacotherapy; the latter (often using selective serotonin reuptake inhibitors) is arguably little better, if at all, than placebo in mild and moderate cases.

Hallucinogens—e.g. psilocin (a dephosphorylated active metabolite of psilocybin), and synthetic hallucinogens related to mescaline-act through a different mechanism and show some promise for treating a variety of mental health conditions including major depression, existential distress associated with a serious illness, post-traumatic stress disorder, and addiction.

Specifically, the serotonin receptor (5-HT2AR) was noted for its importance as a target of serotonergic psychedelic drugs, due to its overexpression in the cortex region compared to other 5-HT receptors. Agonists of the 5-hydroxytryptamine 2A receptor (5-HT2AR) are sought after as potential pharmaceuticals for a variety of neuropsychiatric diseases including but not limited to depression, anxiety, substance abuse, migraine headaches, and/or cluster headaches, and various somatic illnesses including but not limited to various inflammatory, cardiovascular, genitourinary, and/or pain disorders.

Both psilocin and mescaline are agonists of 5-HT2AR. Psilocybin's efficacy in phase II clinical trials of depression and anxiety appears to be both rapid and enduring. Whereas preliminary data suggest that mescaline users report positive improvements in psychiatric conditions, no study has yet explored the possible mechanisms of change related to these psychiatric improvements or assessed the long-term effects associated with mescaline consumption. Despite medicinal chemistry studies of various tryptamines and phenethylamines, the precise broader neurobiological effects of psychedelic-assisted psychotherapy remain unclear.

Research continues into modifying these scaffolds conducting SAR studies, but progress remains slow. Currently, there are 14 ligand-bound structures of 5-HT2AR that have been experimentally determined. The availability of these structures will support the structure-based modeling approach to understand key interactions and conformational dynamics of ligand-receptor systems.

Based on the ligand activity, these ligand-bound structures consist of the following four categories: (a) six agonists, namely, serotonin, psilocin, LSD, 25CN—NBOH, IHCH-7113, and (R)-69 tetrahydropyridine; (b) two partial agonists, namely, lisuride and aripiprazole; (c) three antagonists, namely, lumateperone, zotepine, and cariprazine; (d) two inverse agonist, namely, risperidone and methiothepin (or metitepine). The effects of different psychedelics differ, and this may be due to both the activity and selectivity (off-target effects). These effects may or may not be desirable, but more selective and potent agonists of the receptor would be valuable both as potential next-generation therapeutics, and as useful tools to better understand fundamental neurochemistry and biology and to lead to further therapeutic innovation. New chemical identities could be the basis of a potent and selective psychedelic drug with a better-defined molecular mode of action (current psychedelics interact with multiple receptors to different degrees, potentially explaining why experiences are qualitatively different).

### SUMMARY OF THE INVENTION

The present invention is directed to agonists of 5-HT2AR and a collection of compounds that show particular promise for therapeutic treatment of humans and/or mammals.

In particular, the invention discloses 15 ligands (ligands numbered TTA005, TTA009, TTA011, TTA012, TTA015, TTA019, TTA020, TTA024, TTA031, TTA033, TTA035, TTA036, TTA037, TTA038, TTA039) that should have better calculated binding affinities relative to psilocin. The structures of these ligands are shown below in Table 1.

TABLE 1

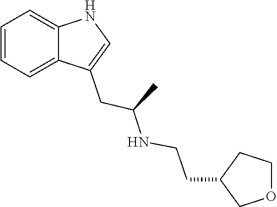 [SMILES: C[C@H](Cc1c[nH]c2ccccc12)NCC[C@@H]1CCOC1]

TTA005

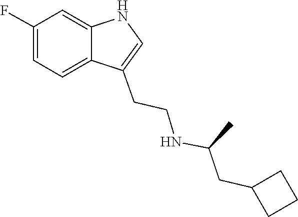 [SMILES: C[C@@H](CC1CCC1)NCCc1c[nH]c2cc(F)ccc12]

TTA009

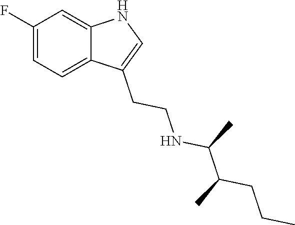 [SMILES: CCC[C@@H](C)[C@H](C)NCCc1c[nH]c2cc(F)ccc12]

TTA011

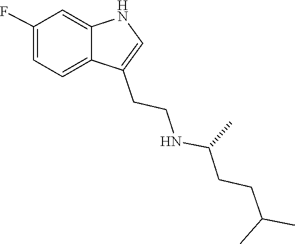 [SMILES: CC(C)CC[C@@H](C)NCCc1c[nH]c2cc(F)ccc12]

TTA012

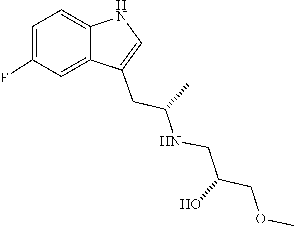 [SMILES: COC[C@H](O)CN[C@@H](C)Cc1c[nH]c2ccc(F)cc12]

TTA015

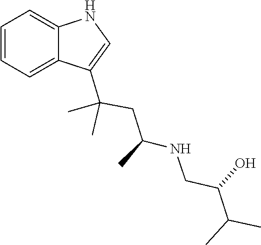 [SMILES: CC(C)[C@@H](O)CN[C@@H](C)CC(C)(C)c1c[nH]c2ccccc12]

TTA019

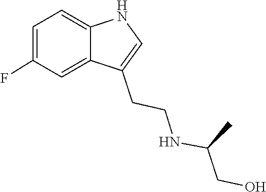 [SMILES: C[C@@H](CO)NCCc1c[nH]c2ccc(F)cc12]

TTA020

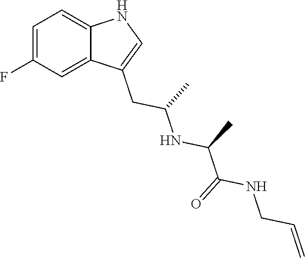 [SMILES: C=CCNC(=O)[C@H](C)N[C@@H](C)Cc1c[nH]c2ccc(F)cc12]

TTA024

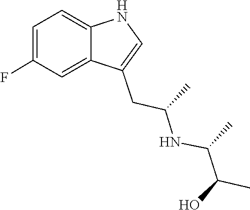 [SMILES: C[C@@H](Cc1c[nH]c2ccc(F)cc12)N[C@H](C)[C@@H](C)O]

TTA031

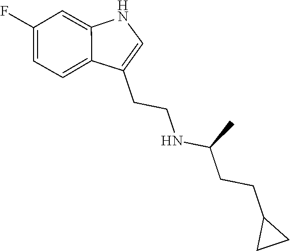 [SMILES: C[C@@H](CCC1CC1)NCCc1c[nH]c2cc(F)ccc12]

TTA033

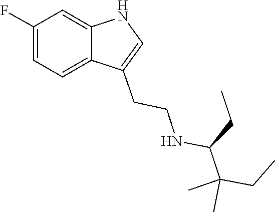 [SMILES: CC[C@H](NCCc1c[nH]c2cc(F)ccc12)C(C)(C)CC]

TTA035

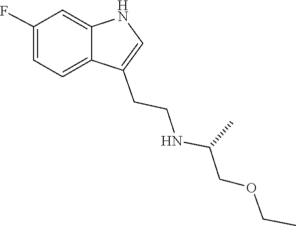 [SMILES: CCOC[C@@H](C)NCCc1c[nH]c2cc(F)ccc12]

TTA036

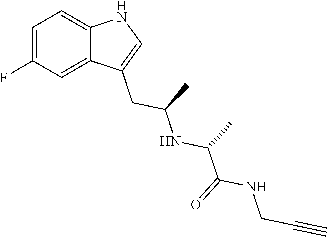 [SMILES: C#CCNC(=O)[C@@H](C)N[C@H](C)Cc1c[nH]c2ccc(F)cc12]

TTA037

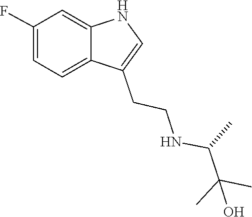 [SMILES: C[C@@H](NCCc1c[nH]c2cc(F)ccc12)C(C)(C)O]

TTA038

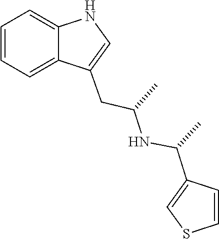 [SMILES: C[C@@H](Cc1c[nH]c2ccccc12)N[C@H](C)c1ccsc1]

TTA039

The preferred ligands shown in Table 1 represent selective and potent agonists of 5-HT2AR that could be pharmacologically useful as therapeutic agents or as potent and selective research tools with a better-defined molecular mode of action that would help better identify the affected molecular receptors.

The ligands of the present invention concern pharmaceutically-acceptable psychedelics, tryptamine analogues and salts thereof. In particular, though not exclusively, the invention relates to formulations and uses of the same as a medicament that, when administered to a human or mammal patient, will convert to an active form in vivo, and act as a 5HT_{2A}R agonist which are useful to treat mental disorders, such as a depressive condition, including unipolar and bipolar depressive conditions, such as, e.g., depression, depression from generalized anxiety, major depression, treatment resistant depression and postpartum depression.

As such, a therapeutically effective composition according to the invention comprises: an active ingredient that includes [1-(1H-indol-3-yl)propan-2-yl][2-(oxolan-3-yl)ethyl]amine (TTA005); (1-cyclobutylpropan-2-yl)[2-(6-fluoro-1H-indol-3-yl)ethyl]amine (TTA009); [2-(6-fluoro-1H-indol-3-yl)ethyl](3-methylhexan-2-yl)amine (TTA011); [2-(6-fluoro-1H-indol-3-yl)ethyl](5-methylhexan-2-yl)amine (TTA012); [1-(5-fluoro-1H-indol-3-yl)propan-2-yl][(2R)-2-hydroxy-3-methoxypropyl]amine (TTA015); 1-{[4-(1H-indol-3-yl)-4-methylpentan-2-yl]amino}-3-methylbutan-2-ol (TTA0-19); 2-{[2-(5-fluoro-1H-indol-3-yl)ethyl]amino}propan-1-ol (TTA020); 2-{[1-(5-fluoro-1H-indol-3-yl)propan-2-yl]amino}-N-(prop-2-en-1-yl)propenamide (TTA024); 3-{[1-(5-fluoro-1H-indol-3-yl)propan-2-yl]amino}butan-2-ol (TTA031); (4-cyclopropylbutan-2-yl)[2-(6-fluoro-1H-indol-3-yl)ethyl]amine (TTA033); (4,4-dimethylhexan-3-yl)[2-(6-fluoro-1H-indol-3-yl)ethyl]amine (TTA035); (1-ethoxypropan-2-yl)[2-(6-fluoro-1H-indol-3-yl)ethyl]amine (TTA036); 2-{[1-(5-fluoro-1H-indol-3-yl)propan-2-yl]amino}-N-(prop-2-yn-1-yl)propenamide (TTA037); 3-{[2-(6-fluoro-1H-indol-3-yl)ethyl]amino}-2-methylbutan-2-ol (TTA038); [1-(1H-indol-3-yl)propan-2-yl][1-(thiophen-3-yl)ethyl]amine (TTA039), their pharmaceutically acceptable salts, or diastereomers thereof.

The present invention is also directed to a therapy process that comprises administering to a human or mammal subject in need thereof a therapeutically-effective amount of the above therapeutically effective composition.

In another aspect, the invention comprises a method of treating a mental disorder, comprising the step of administering an effective amount of a ligand described herein. In some embodiments, the mental disorder is a depressive condition, including unipolar and bipolar depressive conditions, such as but not limited to depression, depression from generalized anxiety, major depression, treatment resistant depression and postpartum depression.

The invention provides for the treatment and/or prevention of psychiatric disorders, and/or neurological disorders, and/or degenerative disorders, and/or inflammatory disorders. In another aspect, the invention relates to the use of a compound described herein to treat a mental disorder, or in the manufacture of a medicament for treating a mental disorder, such as depression.

The general method of treatment comprises administering to a human or mammal subject in need thereof a therapeutically-effective amount of an acceptable psychedelic analogue in one or more pharmaceutically acceptable carriers or excipients.

The administered composition is formulated for oral, sublingual, intranasal, pulmonary administration, buccal, sublingual, rectal, transdermal, transmucosal, epidural, intrathecal, intraocular topical, creams, lotions, gels and eye drops using one or more excipients that are traditionally used in such formulations.

### DETAILED DESCRIPTION OF THE INVENTION

The term “neurological disorders” refers to any structural, biochemical and/or electrical abnormalities in the brain, spinal cord or other nerves and includes neurodevelopment and neurodegenerative diseases that may benefit from of neural plasticity modulation. In a preferred embodiment, the term “neurological disorder” refers to one or more disorders selected from the following acquired brain injury, ataxia brain tumor, dementia, dystonia epilepsy, temporal lobe epilepsy, pain associated with neurological disorders, headache disorders, functional and dissociative neurological symptoms, neuroinfections, meningitis, disorders associated with malnutrition, motor neuron disease, multi-system atrophy, multiple sclerosis, amyotrophic lateral sclerosis, mesial temporal lobe hippocampal sclerosis, muscular dystrophy, myalgic encephalomyelitis, Parkinson's disease, progressive supranuclear palsy, cerebral palsy, Huntington's disease, Alzheimer's disease, frontal lobe dementia, vascular dementia, dementia with Lewy bodies, mild cognitive impairment (MCI) associated with aging and chronic disease and its treatment, including chemotherapy, immunotherapy and radiotherapy, mild corticobasal degeneration, disorders associated with accumulation of beta amyloid, and/or with the accumulation or disruption of tau protein and its metabolites. Lyme encephalopathy, toxic encephalopathy, cognitive decline associated with aging, spinabifida, hydrocephalus, spinal injury, stroke, Tourette syndrome, and transverse myelitis, corticobasal degeneration, supranuclear palsy, epilepsy; Nervous System trauma, Nervous System infections, Nervous System inflammation, including inflammation from autoimmune disorders, including NMDAR encephalitis, and cytopathology from toxins, (including microbial toxins, heavy metals, and pesticides etc.), stroke, multiple sclerosis, Huntington's disease, mitochondrial disorders, Fragile X syndrome, Angelman syndrome, hereditary ataxias, neuro-otological and eye movement disorders, amyotrophic lateral sclerosis, tardive dyskinesias (TD), hyperkinetic disorders; attention deficit hyperactivity disorder and attention deficit disorders; restless leg syndrome, autism spectrum disorders, tuberous sclerosis, Rett syndrome, cerebral palsy, disorders of the reward system including eating disorders [including anorexia nervosa (“AN”) and bulimia nervosa (“BN”), and binge eating disorder (“BED”), trichotillomania, dermotillomania, nail biting, migraine, fibromyalgia, and peripheral neuropathy of any etiology. Symptoms or manifestations of nervous system disorders that may be treated or prevented by neuroplastogen substances and drugs include, a decline, impairment, or abnormality in cognitive abilities including executive function, attention, cognitive speed, memory, language functions (speech, comprehension, reading and writing), orientation in space and time, praxis, ability to perform actions, ability to recognize faces or objects, concentration, and alertness; abnormal movements including akathisia, bradykinesia, tics, myoclonus, dyskinesias, including dyskinesias relate to Huntington's disease, levodopa induced dyskinesias and neuroleptic induced dyskinesias, dystonias, tremors, including essential tremor, and restless leg syndrome; parasomnias, insomnia, disturbed sleep pattern; psychosis; delirium; agitation; headache; motor weakness, spasticity, impaired physical endurance; sensory impairment, including impairment of vision and visual field defects, smell, taste, hearing and balance, and dysesthesias; dysautonomia; and ataxia, impairment of balance or coordination, tinnitus, neuro-otological and eye movement impairments, neurological symptoms of alcohol withdrawal, including delirium, headache, tremors, hallucinations, hypertension.

The term “degenerative disorders” refers to one or more disorders selected from the following degenerative disorders, neurodegenerative diseases of the retina like glaucoma, diabetic retinopathy and age-related macular degeneration, retinitis pigmentosa, Usher disease and Bardet-Biedl syndrome, motor neuron disease, prion disease, spinocerebelluar ataxia and apathy syndrome.

The term “inflammatory disorders” refers to one or more disorders selected from the following inflammatory disorders, of atherosclerosis, asthma, rheumatoid arthritis, psoriasis, type II diabetes, irritable bowel syndrome, Crohn's disease, septicemia, depression, schizophrenia, multiple sclerosis, conjunctivitis, Alzheimer's disease, chronic obstructive pulmonary disease, neuro-inflammation, metabolic syndrome, impaired glucose tolerance, non-alcoholic fatty liver disease (NAFLD), (NAFL) and their complications, nonalcoholic steatohepatitis (NASH) and conjunctivitis.

The general method of treatment comprises administering to a human or mammal subject in need thereof a therapeutically-effective amount of an acceptable psychedelic analogue in one or more pharmaceutically acceptable carriers or excipients.

The administered composition is formulated for oral, sublingual, intranasal, pulmonary administration, buccal, sublingual, rectal, transdermal, transmucosal, epidural, intrathecal, intraocular topical, creams, lotions, gels and eye drops using one or more excipients that are traditionally used in such formulations.

Embodiments of the present invention comprise novel synthetic psychedelic prodrugs. The prodrugs may be useful for treatment of mental disorders such as depression, including without limitation, major depression, treatment resistant depression and postpartum depression. As used herein, the term “mental disorder” includes those disorders which may be diagnosed by a mental health professional as a psychological or psychiatric disorder, including those which may be diagnosed by reference to Diagnostic and Statistical Manual of Mental Disorders (DSM-5).

The term “treating”, “treat” or “treatment” as used herein embraces both preventative, i.e., prophylactic, and palliative treatment, i.e., relieve, alleviate, or slow the progression of the patient's disease, disorder or condition.

As used herein, “psychedelic state” is an altered state of consciousness experienced by a person, which may include intensified sensory perception, perceptual distortion or hallucinations, and/or feelings of euphoria or despair. Psychedelic states have been described as resulting from psychedelic drugs such as DMT (dimethyltryptamine), LSD, mescaline or psilocybin. Other known psychedelic drugs include the 4-hydroxy analogs of N-Methyl-N-isopropyltryptamine (MiPT) and N,N-diisopropyltryptamine (DiPT).

The present invention comprises prodrugs of 5HT_{2A}R agonists which induce a psychedelic state or which still provide a beneficial therapeutic effect without being associated with a psychedelic state. The prodrugs may be used in combination with other treatments known to be effective for treating mental disorders, such as psychotherapy, electroconvulsive therapy and/or other pharmaceutical compounds, for example, with concomitant use of tricyclic antidepressants (TCAs), selective serotonin reuptake inhibitors (SSRIs), selective norepinephrine reuptake inhibitors (SNRIs), monoamine oxidase inhibitors (MOAIs) or other anti-depressants. In preferred embodiments, the treatment may produce lasting effects, for example longer than 1 month after a single treatment, preferably longer than 3 months, and more preferably longer than 6 months. In some embodiments, additional therapy may not be required.

“Compounds” when used herein includes any pharmaceutically acceptable derivative or variation, including conformational isomers (e.g., cis and trans isomers) and all optical isomers (e.g., enantiomers and diastereomers), racemic, diastereomeric and other mixtures of such isomers, as well as solvates, hydrates, isomorphs, polymorphs, tautomers, esters, salt forms, and prodrugs. The expression “prodrug” refers to compounds that are drug precursors which following administration, release the drug (or “active”) in vivo via some chemical or physiological process (e.g., hydrolysis, enzymatic cleavage or hydrolysis, or metabolism is converted to the desired drug form). The invention includes within its scope the pharmaceutically acceptable salts of the compounds of the invention. Accordingly, the phrase “or a pharmaceutically acceptable salt thereof” is implicit in the description of all compounds described herein unless explicitly indicated to the contrary.

In some embodiments, the compounds of the present invention comprise prodrug compounds that are readily purified, formulated and stable, and preferably may be used to provide highly soluble drug substances, with fast onset and elimination for convenient use in a clinical setting. In some embodiments, the compounds may be produced as a zwitterion, which may be converted to a pharmaceutically acceptable salt.

In some embodiments, the compounds of the present invention preferably allow for fast cleavage in vivo of the prodrug moiety to give the active pharmacophore, for example, 90% conversion may occur in under 4 hours, preferably in less than 2 hours, and more preferably in less than 1 hour. Prodrugs may have lesser, little or no pharmacological activity themselves, however when administered to a patient, may be converted into an active compound, for example, by hydrolytic cleavage.

“Alkyl,” by itself or as part of another substituent, refers to a saturated branched, straight-chain or cyclic monovalent hydrocarbon radical derived by the removal of one hydrogen atom from a single carbon atom of a parent alkane. The term “alkyl” includes cycloalkyl. Typical alkyl groups include, but are not limited to, methyl; ethyl; propyls such as propan-1-yl, propan-2-yl (isopropyl), cyclopropan-1-yl, etc.; butanyls such as butan-1-yl, butan-2-yl (sec-butyl), 2-methyl-propan-1-yl (isobutyl), 2-methyl-propan-2-yl (t-butyl), cyclobutan-1-yl, etc.; and the like. In some embodiments, an alkyl group comprises from 1 to 20 carbon atoms (C1-C20 alkyl). In other embodiments, an alkyl group comprises from 1 to 10 carbon atoms (C1-C10 alkyl). In still other embodiments, an alkyl group comprises from 1 to 6 carbon atoms (C1-C6 alkyl) or 1 to 4 carbon atoms (C1-C4). C1-C6 alkyl is also known as “lower alkyl”.

The term “arylalkyl” is a term of the art and as used herein refers to an alkyl group, for example a C1-6 alkyl group, substituted with an aryl group, where the residue is linked to the main molecule through the alkyl group. An example of arylalkyl is the benzyl group, that is, the phenyl-methyl group.

“Substituted,” when used to modify a specified group or radical, means that one or more hydrogen atoms of the specified group or radical are each, independently of one another, replaced with the same or different substituent(s). The term “substituted” specifically envisions and allows for one or more substitutions that are common in the art. However, it is generally understood by those skilled in the art that the substituents should be selected so as to not adversely affect the useful characteristics of the compound or adversely interfere with its function.

The term “optionally substituted” denotes the presence or absence of the substituent group(s). That is, it means “substituted or unsubstituted”. For example, optionally substituted alkyl includes both unsubstituted alkyl and substituted alkyl. The substituents used to substitute a specified group can be further substituted, typically with one or more of the same or different groups selected from the various groups specified above.

The term “psychiatric disorders” refers to one or more disorders selected from the following psychiatric disease as defined as defined by DMS5 and ICD11 that may benefit from modulation of neural plasticity, including Schizophrenia spectrum and other psychotic disorders, Bipolar and related disorders, Depressive disorders, COVID Depressive disorder, generalized anxiety disorders, Obsessive-compulsive and related disorders, Trauma- and stressor-related disorders, Dissociative disorders, Somatic symptom and related disorders, Feeding and eating disorders, Elimination disorders, Sleep-wake disorders, Sexual Disruptive, impulse-control, and conduct disorders, Substance-related and addictive disorders, panic disorder, agoraphobia, social anxiety disorder, phobias, posttraumatic stress disorder, obsessive compulsive disorder, generalized anxiety disorder, anorexia nervosa, binge eating disorder, bulimia nervosa, psychosis, schizophrenia, substance addiction and personality disorders, neurocognitive disorders, personality disorders, paraphilic disorders and for the reduction of suicidal ideation in a patient suffering from a life-threatening disease.

The term “neurological disorders” refers to any structural, biochemical and/or electrical abnormalities in the brain, spinal cord or other nerves and includes neurodevelopment and neurodegenerative diseases that may benefit from of neural plasticity modulation. In a preferred embodiment, the term “neurological disorder” refers to one or more disorders selected from the following acquired brain injury, ataxia brain tumor, dementia, dystonia epilepsy, temporal lobe epilepsy, pain associated with neurological disorders, headache disorders, functional and dissociative neurological symptoms, neuroinfections, meningitis, disorders associated with malnutrition, motor neuron disease, multi-system atrophy, multiple sclerosis, amyotrophic lateral sclerosis, mesial temporal lobe hippocampal sclerosis, muscular dystrophy, myalgic encephalomyelitis, Parkinson's disease, progressive supranuclear palsy, cerebral palsy, Huntington's disease, Alzheimer's disease, frontal lobe dementia, vascular dementia, dementia with Lewy bodies, mild cognitive impairment (MCI) associated with aging and chronic disease and its treatment, including chemotherapy, immunotherapy and radiotherapy, mild corticobasal degeneration, disorders associated with accumulation of beta amyloid, and/or with the accumulation or disruption of tau protein and its metabolites. Lyme encephalopathy, toxic encephalopathy, cognitive decline associated with aging, spinabifida, hydrocephalus, spinal injury, stroke, Tourette syndrome, and transverse myelitis, corticobasal degeneration, supranuclear palsy, epilepsy; Nervous System trauma, Nervous System infections, Nervous System inflammation, including inflammation from autoimmune disorders, including NMDAR encephalitis, and cytopathology from toxins, (including microbial toxins, heavy metals, and pesticides etc.), stroke, multiple sclerosis, Huntington's disease, mitochondrial disorders, Fragile X syndrome, Angelman syndrome, hereditary ataxias, neuro-otological and eye movement disorders, amyotrophic lateral sclerosis, tardive dyskinesias (TD), hyperkinetic disorders; attention deficit hyperactivity disorder and attention deficit disorders; restless leg syndrome, autism spectrum disorders, tuberous sclerosis, Rett syndrome, cerebral palsy, disorders of the reward system including eating disorders [including anorexia nervosa (“AN”) and bulimia nervosa (“BN”), and binge eating disorder (“BED”), trichotillomania, dermotillomania, nail biting, migraine, fibromyalgia, and peripheral neuropathy of any etiology. Symptoms or manifestations of nervous system disorders that may be treated or prevented by neuroplastogen substances and drugs include, a decline, impairment, or abnormality in cognitive abilities including executive function, attention, cognitive speed, memory, language functions (speech, comprehension, reading and writing), orientation in space and time, praxis, ability to perform actions, ability to recognize faces or objects, concentration, and alertness; abnormal movements including akathisia, bradykinesia, tics, myoclonus, dyskinesias, including dyskinesias relate to Huntington's disease, levodopa induced dyskinesias and neuroleptic induced dyskinesias, dystonias, tremors, including essential tremor, and restless leg syndrome; parasomnias, insomnia, disturbed sleep pattern; psychosis; delirium; agitation; headache; motor weakness, spasticity, impaired physical endurance; sensory impairment, including impairment of vision and visual field defects, smell, taste, hearing and balance, and dysesthesias; dysautonomia; and ataxia, impairment of balance or coordination, tinnitus, neuro-otological and eye movement impairments, neurological symptoms of alcohol withdrawal, including delirium, headache, tremors, hallucinations, hypertension.

The term “degenerative disorders” refers to one or more disorders selected from the following degenerative disorders, neurodegenerative diseases of the retina like glaucoma, diabetic retinopathy and age-related macular degeneration, retinitis pigmentosa, Usher disease and Bardet-Biedl syndrome, motor neuron disease, prion disease, spinocerebelluar ataxia and apathy syndrome.

The term “inflammatory disorders” refers to one or more disorders selected from the following inflammatory disorders, of atherosclerosis, asthma, rheumatoid arthritis, psoriasis, type II diabetes, irritable bowel syndrome, Crohn's disease, septicemia, depression, schizophrenia, multiple sclerosis, conjunctivitis, Alzheimer's disease, chronic obstructive pulmonary disease, neuro-inflammation, metabolic syndrome, impaired glucose tolerance, non-alcoholic fatty liver disease (NAFLD), (NAFL) and their complications, nonalcoholic steatohepatitis (NASH) and conjunctivitis.

### Formulations and Compositions

The invention also provides pharmaceutically acceptable compositions which comprise a therapeutically effective amount of one or more of the compounds described herein, formulated together with one or more pharmaceutically acceptable carriers (additives) and/or diluents, and optionally, one or more additional therapeutic agents. While it is possible for a compound described herein to be administered alone, it is preferable to administer the compound as a pharmaceutical composition.

The term “pharmaceutical composition” means a composition comprising a compound of the invention in combination with at least one additional pharmaceutically acceptable carrier.

A “pharmaceutically acceptable carrier” refers to media generally accepted in the art for the delivery of biologically active agents to animals, in particular, mammals, including, i.e., adjuvant, excipient or vehicle, such as diluents, osmotic complement, preserving agents, fillers, flow regulating agents, disintegrating agents, wetting agents, emulsifying agents, suspending agents, sweetening agents, flavoring agents, perfuming agents, antibacterial agents, antifungal agents, lubricating agents, polymers, solubilizing agents, stabilizers, antioxidants and dispensing agents, depending on the nature of the mode of administration and dosage forms. Each carrier must be “acceptable” in the sense of being compatible with the other ingredients of the formulation and not injurious to the patient.

As used herein, “oral” administration includes swallowing for ingestion in the stomach or gut, and further includes lingual, sublingual, buccal and oropharyngeal administration. The compounds of this invention can be administered for any of the uses or methods described herein by any suitable means, for example, orally, such as tablets, capsules (each of which may include sustained release or timed release formulations), pills, powders, granules, elixirs, suspensions (including nano suspensions, micro suspensions, spray-dried dispersions), syrups, and emulsions; sublingually (e.g. as thin films, effervescent tablets or tablets that dissolve spontaneously under the tongue); parenterally, such as by subcutaneous, intravenous, intramuscular injection, or infusion techniques (e.g., as sterile injectable aqueous or non-aqueous solutions or suspensions); nasally, including administration to the nasal membranes, such as by inhalation spray; or rectally such as in the form of suppositories.

The dosage regimen for the compounds described herein will, of course, vary depending upon known factors, such as the pharmacokinetic and pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient; and, the effect desired. The selected dosage level may also depend on the additional factors including the activity of the particular compounds and pharmaceutical compositions described herein, whether an ester, salt or amide substituent is of the compound is used, the time of administration, the rate of excretion or metabolism of the particular compound being employed, the rate and extent of absorption, the duration of the treatment, other drugs that may be administered to the patient, compounds and/or materials used in combination with the particular compound employed and like factors well known in the medical arts.

Generally, the dosage of the prodrug for a therapy session, when used for the indicated effects, will range between about 0.001 to about 500 mg per dose, preferably between about 0.01 to about 200 mg per dose, and most preferably between about 0.1 to about 50 mg per dose, such as 10, 20, 30, 40, 50, 100 or 200 mg. Intravenously, the most preferred doses will range from about 0.01 to about 10 mg/kg/minute during a constant rate infusion.

Compounds of this invention may be administered in a single daily dose, or the total daily dosage may be administered in multiple divided doses, such as two, three, or four times daily. Alternatively, the doses may be provided on a weekly, biweekly, or monthly basis. In a preferred embodiment, only one or two doses are required for an anti-depressant effect that may extend for 1, 2, 3 or 6 months, or more.

For tablet dosage forms, depending on dose, the drug may make up from 1 wt % to 80 wt % of the dosage form, more typically from 5 wt % to 60 wt % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl substituted hydroxypropyl cellulose, starch, pregelatinized starch and sodium alginate. Generally, the disintegrant will comprise from 1 wt % to 25 wt %, preferably from 5 wt % to 20 wt % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinized starch, hydroxypropyl cellulose and hydroxypropyl methylcellulose. Tablets may also contain diluents, such as lactose (monohydrate, spray dried monohydrate, anhydrous and the like), mannitol, xylitol, dextrose, sucrose, sorbitol, microcrystalline cellulose, starch and dibasic calcium phosphate dihydrate.

Tablets may also optionally include surface active agents, such as sodium lauryl sulfate and polysorbate 80, and glidants such as silicon dioxide and talc. When present, surface active agents are typically in amounts of from 0.2 wt % to 5 wt % of the tablet, and glidants typically from 0.2 wt % to 1 wt % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally are present in amounts from 0.25 wt % to 10 wt %, preferably from 0.5 wt % to 3 wt % of the tablet.

Other conventional ingredients include anti-oxidants, colorants, flavoring agents, preservatives and taste masking agents.

Exemplary tablets contain up to about 80 wt % drug, from about 10 wt % to about 90 wt % binder, from about 0 wt % to about 85 wt % diluent, from about 2 wt % to about 10 wt % disintegrant, and from about 0.25 wt % to about 10 wt % lubricant.

Tablet blends may be compressed directly or by roller to form tablets. Tablet blends or portions of blends may alternatively be wet, dry, or melt granulated, melt congealed, or extruded before tableting. The final formulation may include one or more layers and may be coated or uncoated; or encapsulated.

The formulation of tablets is discussed in detail in “Pharmaceutical Dosage Forms: Tablets, Vol. 1”, by H. Lieberman and L. Lachman, Marcel Dekker, N.Y., N.Y., 1980 (ISBN 0 8247 6918 X), the disclosure of which is incorporated herein by reference in its entirety.

A typical capsule for oral administration contains at least one of the compounds of the present invention (e.g. 25 mg), lactose (e.g. 75 mg), and magnesium stearate (e.g. 15 mg). The mixture is passed through a 60 mesh sieve and packed into a No. 1 gelatin capsule.

Liquid formulations include suspensions, solutions, syrups and elixirs. Such formulations may be used as fillers in soft or hard capsules and typically include a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular, and subcutaneous. Suitable devices for parenteral administration include needle (including micro needle) injectors, needle free injectors and infusion techniques.

Parenteral formulations are typically aqueous solutions which may contain excipients such as salts, carbohydrates and pH adjusting or buffering agents (preferably to a pH of from 3.0 and 7.0, preferably 4.0 to 6.0, and more preferably 4.5 to 5.5), but, for some applications, they may be more suitably formulated as a sterile non aqueous solution or as a dried form to be used in conjunction with a suitable vehicle such as sterile, pyrogen free water or pre-fabricated, ready-to-mix aqueous buffer. Osmotic agents may be included to control tonicity.

The preparation of parenteral kits for reconstitution at point-of-care under sterile conditions, for example, by lyophilization, may readily be accomplished using standard pharmaceutical techniques well known to those skilled in the art.

A typical injectable preparation is produced by aseptically placing at least one of the compounds of the present invention (e.g. 25 mg) into a vial as a sterile filtered solution, aseptically freeze-drying and scaling. For use, the contents of the vial are mixed with e.g. 2 mL of physiological saline for injection, optionally with an appropriate amount of osmotic complements and pH adjusters to achieve a slightly acidic to neutral pH (e.g. pH 4-7), to produce an injectable preparation with low irritation but retain solubility and/or stability of the prodrug.

Compounds of the invention may be combined with soluble macromolecular entities, such as cyclodextrin and suitable derivatives thereof or polyethylene glycol containing polymers, in order to improve their solubility, dissolution rate, taste masking, bioavailability and/or stability for use in any of the aforementioned modes of administration.

Drug cyclodextrin complexes, for example, are found to be generally useful for most dosage forms and administration routes. Both inclusion and non-inclusion complexes may be used. As an alternative to direct complexation with the drug, cyclodextrin may be used as an auxiliary additive, i.e. as a carrier, diluent, or solubilizer. The materials most commonly used for these purposes are alpha, beta and gamma cyclodextrins, examples of which may be found in PCT Publication Nos. WO 91/11172, WO 94/02518 and WO 98/55148, the disclosures of which are incorporated herein by reference in their entireties.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art. Actual dosage levels of the active ingredients in the pharmaceutical compositions of this invention may be varied so as to obtain an amount of the active ingredient which is effective to achieve the desired therapeutic response for a particular patient, composition, and mode of administration.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required to achieve the desired therapeutic effect and gradually increase the dosage until the desired effect is achieved.

In general, a suitable daily dose of a compound of the invention will be an amount of the compound which is the lowest dose effective to produce a therapeutic effect. Such an effective dose will generally depend upon the factors described above.

As used herein, a “therapeutically effective amount” refers to that amount of a compound being administered which will relieve to some extent one or more of the symptoms of the disorder being treated. In reference to the treatment of depression, a therapeutically effective amount refers to that amount which has the effect of reducing the severity of depression. Depression severity may be assessed using well-known structured assessment tools such as Structured Clinical Interview for DSM-5 (SCID-5) and the GRID-Hamilton Depression Rating Scale (GRID-HAMD). A therapeutically effective amount may be less than that required for a psychedelic state.

An effective dosage can be administered in one or more administrations. For the purposes of this invention, an effective dosage of drug, compound, or pharmaceutical composition is an amount sufficient to accomplish prophylactic or therapeutic treatment either directly or indirectly. As is understood in the clinical context, an effective dosage of drug, compound or pharmaceutical composition may or may not be achieved in conjunction with another therapy, drug, compound, or pharmaceutical composition.

### Therapeutic Methods and Uses

Treatment with the novel prodrugs of the present invention may substantially alleviate clinical or subclinical depression and may avoid relapse, particularly if used in combination with psychotherapy for the treatment of depression. It is known that administration of an effective dose of psilocybin produced rapid and large reductions in depressive symptoms, and many subjects achieve remission through a four-week follow up (Davis et. al.) Without restriction to a theory, it is believed that the psychedelic state is associated with the beneficial effects, however, some compounds which are 5HT_{2A}R agonists may provide the desired therapeutic effect without the psychedelic state. One aspect of the invention comprises prodrugs of those 5HT_{2A}R agonists which do provide a beneficial therapeutic state.

In general, the present invention includes the use of a compound of the present invention herein, to treat any disease or disorder which may be alleviated by a 5HT_{2A}R agonist, or the use of a compound of the present invention herein to manufacture a medicament to treat any disease or disorder which may be alleviated by a 5HT_{2A}R agonist, or a method of treating any disease or disorder which may be alleviated by a 5HT_{2A}R agonist.

In some embodiments, the invention may comprise the use of the compounds of the present invention to treat mental disorders. In some embodiments, the invention may comprise the use of the compounds of the present invention to treat depression, and particularly drug resistant depression. Other conditions that may be treated include: anxiety disorders, including anxiety in advanced stage illness e.g. cancer as well as generalized anxiety disorder, depression including major depressive disorder, postpartum depression, cluster headaches, obsessive compulsive disorder, personality disorders including conduct disorder, drug disorders including: alcohol dependence, nicotine dependence, opioid dependence, cocaine dependence and other addictions including gambling disorder, eating disorder and body dysmorphic disorder, chronic pain, or chronic fatigue.

In some embodiments, the invention may comprise a method of treating mental disorders comprising administering to a subject in need thereof a therapeutically effective amount of a compound of the present invention. In one embodiment, there is provided a method of treating depression by administering to a subject in need thereof therapeutically effective amount of a compound of the present invention. The depression effects may be drug-resistant depression or major depressive disorder.

For example, a patient diagnosed with depression may be screened prior to treatment, and then prepared for a dosing session by a trained psychotherapist. Within a dosing session, a compound of the present invention may be administered by injection of a sterile solution at a rate of 0.01-0.3 mg/kg to the patient. The patient is preferably seated for the duration of the session while being blindfolded. For safety, a trained health care professional may monitor the patient throughout the dosing session, which may last up to 12 hours. In some cases, music may be played for the patient. When the health care professional can determine that the drug substance has cleared, the psychotherapist may assist the patient with any questions relating to the psychedelic experience, and then the patient may be discharged.

To further alleviate any anxiety that may occur relative to therapy, the physician may prefer to divide the therapeutic dose and thereby reduce the initial onset of psychoactivity before applying the full complement of the dosage to achieve the full effect.

In some embodiments, treatment with a compound of the present invention may be combined with concomitant treatment with another anti-depressant drugs, either concurrently or consecutively. In preferred embodiments, treatment with a compound of the present invention is combined with psychotherapy, which may be applied prior to or after treatment. If prior to, the session may focus the patient on the intent of treatment. If after, psychotherapy is preferably performed within 48 hours of the dosing session to help the patient integrate any feelings, emotions, visions or thoughts that may have occurred during the session, as well as to allow the psychotherapist may offer advice on how best to change thinking or behavior patterns so as to improve anti-depression outcomes. Psychotherapy may continue as needed after the dosing session, for example, up to an additional 3 months, to help the patient integrate any experiences or learnings that occurred to the patient during the dosing session.

### Example 1: Virtual Screening

We employed a target analysis workflow (target validation step) for all ligand-bound structures to choose a suitable structure for the virtual screening campaign. Before building up model systems, we found steric clashes and missing atoms in the protein reliability reports of all PDB structures. These structures were prepared using the Protein Preparation Wizard (Schrödinger Release 2022-3: Protein Preparation Wizard; Epik, Schrödinger, LLC, New York, NY, 2021; Impact, Schrödinger, LLC, New York, NY; Prime, Schrödinger, LLC, New York, NY, 2021) to resolve clashes and missing atoms. This only became recently possible with the publication of crystal structures of this receptor.

Protonation states of titratable residues were assigned using PROPKA (the online version can be found at https://www.ddl.unimi.it/vegaol/propka.htm). After protein preprocessing steps, the reliability reports for all proteins show larger steric clashes; however, these are from crystal mates, which are not biologically relevant. Moreover, there are some residues with alternative positions, which could be resolved by plotting electron density maps of proteins. Therefore, we have run PrimeX of Schrodinger software and ensured that sidechain residues with alternative positions fit the experimental electron density very well. After the protein optimization step, we found some tautomerization (HSD to HSE) and sidechain flips, but these changes in residues were identified to be reasonable based on their interaction environment. Then, we run the virtual screening campaign for the hit identification step. Furthermore, the region corresponding to the binding interface was relaxed in implicit lipid membrane environment (the RMSD between the relaxed structure and the starting agonist-bound X-ray structure was ˜0.1 Å over Ca atoms). The lowest energy relaxed 5-HT2AR structure was selected as a representative model to execute virtual screening.

For docking library preparation, the compounds were filtered based on the following properties: Lipinski Rule of Five less than two violations, predicted permeability in Caco-2 and MDCK cells more than 300 nm/s, predicted IC50 value for the blockage of human ether-a-go-go related gene potassium ion channel value set to more than −5.5 and predicted central nervous system (CNS) activity values included between zero and two. In addition, PAINS (pan-assay interference compounds) were excluded with KNIME's structure filter.

We employed multiple structure-based (SB) and ligand-based (LB) virtual screening campaigns to search for more potent and specific 5-HT2AR agonists. Briefly, we filtered an ultra-huge molecular library (about 30 billion compounds) for molecules showing several drug-like parameters, i.e., compounds with favorable cell permeability, acceptable aqueous solubility, and elimination of compounds with potential toxicophores.

The combined integration of SBVS and LBVS techniques are a promising strategy when data about both the structure of ligand-target complexes and similarity relationships to active compounds are available, leading to a holistic framework suitable to enhance the success of drug discovery projects. The present approach assisted us in both ways, i) designing a targeted structure-based modeling approach, and ii) understanding key interactions and conformational dynamics of ligand-receptor systems. As of this writing, we have successfully implemented pharmacophore similarity screening (based on the fingerprints of dimethyltryptamine) and partially structure-based screening (considering all residues in the binding pocket). The Glide high-throughput virtual screening (HTVS) mode was utilized, subsequent re-docking using standard precision (SP) and extra-precision (XP) mode, followed by an advanced energy approximation (calculations using the solvation model for the top-ranked compounds taken from the previous step), MD simulations, and FEP binding affinity calculations.

One of the main goals of computer-aided drug design is the ability to predict the selectivity and affinity of a drug candidate accurately and reliably towards a target. Alchemical free energy perturbation (FEP) calculations are particularly well suited for this, providing a theoretically rigorous way of computing ligand binding energies of protein targets. Herein, the binding free energies of selected top hits identified from pharmacophore similarity screening were calculated using alchemical FEP calculations following the protocol reported by Jiang et al. All MD simulations and FEP calculations were conducted using NAMD2 and AMBER simulation package. The force field of the top ligands was parameterized using CHARMM general force field (CGenFF) (in NAMD), and general Amber force field (GAFF) (in AMBER). A homogeneous 1-palmitoyl-2-oleoyl-sn-glycero-3-phosphocholine (POPC) membrane bilayer (76 lipids in each leaflet) was built for all receptor-ligand complexes.

The TIP3P water model was used to describe water molecules in a simulation cell with dimensions of (80,80,124) Å³ along the (x,y,z) axes, respectively. Simulations were performed under periodic boundary conditions at a constant temperature of 298.15 K and pressure of 1 atm using Langevin dynamics and the Langevin piston method, respectively. Energy minimization calculations were performed on the membrane protein-ligand model systems, followed by equilibration runs of 20 ns at a 2 femtosecond step.

The Particle Mesh Ewald (PME) method was used to describe long-range interactions and a cut-off distance of 12 Å was used for the Lennard-Jones interactions. Covalent bonds involving hydrogen atoms were constrained using the SHAKE algorithm.

The FEP MD simulations were performed in 16 replica-exchange windows for both (the ligand in bulk solution) and (in the membrane protein model system), yielding an aggregate total of 320 ns FEP simulation time for each membrane protein-ligand complex. For the top hits identified from structure-based methods, a production run of 50 ns was to see the initial stability and then extended to 0.3 μs.

Pharmacophore similarity screening: The rigorous protocol successfully discovered top hits that revealed a consistent interaction profile similar to known actives published in the literature. All top-ranked compounds were posed close to the PIF motif, established strong hydrogen bound with Asp155 and displayed consistent interaction with the toggle switch (Trp336) in conjunction with other conserved interactions. Trp336 (in 5-HT2AR) and the PIF motif have been shown to undergo conformational changes upon receptor activation and these residues were found to be essential for selective agonist potency.

Our comprehensive in silico study allows us to pick only the best-fitting molecules for synthesis and in vitro testing. We identified 15 out of 40 ligands (ligands numbered TTA005, TTA009, TTA011, TTA012, TTA015, TTA019, TTA020, TTA024, TTA031, TTA033, TTA035, TTA036, TTA037, TTA038, TTA039) that have better binding relative to psilocin. Their structures are shown in Table 1 above. Their binding strength relative to psilocin are shown in the table below.

Table 2 shows the sorted, in silico, results from the most promising candidate hits from pharmacophoric screening using dimethyltryptamine as a query molecule. The FEP binding affinities (kcal/mol) are calculated relative to the psilocin compound (column G) such that the more negative value means the ligand is more potent than psilocin. Some virtual hits identified from preliminary similarity/substructure-based screening are also displayed. This virtual screening was performed using Glide workflow and AutoDock vina suit.

In general, the preferred ligand compounds of the invention have a calculated FEP binding affinity relative to psilocin that is within the range from about −14.3 to about −3.2. The calculated FEP binding affinity relative to psilocin is more preferably within the range of −14.3 to −5.5 and most preferably within the range of −14.3 to about −9.3.

TABLE 2

| Ligand # | Enamine ID | G | Variance (+/−) |
|---|---|---|---|
| TTA006 | m_269862bba_17551542_9779938 | * | * |
| TTA013 | m_269862bba_14316030_9779938 | * | * |
| TTA030 | m_269862bba_14650196_9779938 | * | * |
| TTA039 | m_270004bea_9866504_14765758 | −14.3 | 0.4 |
| TTA009 | m_269862bba_9142334_7201654 | −10.9 | 1.2 |
| TTA015 | m_273610aaa_13913088_11516246 | −10.8 | 2.1 |
| TTA037 | m_44daa_13913046_287590 | −10.7 | ′.9 |
| TTA035 | m_269862bba_9142334_12864910 | −9.3 | 1.9 |
| TTA038 | m_269862bba_9142334_14430198 | −6.3 | ′.3 |
| TTA033 | m_269862bba_9142334_8901780 | −5.8 | 0.8 |
| TTA012 | m_269862bba_9142334_7200192 | −5.7 | 1.3 |
| TTA011 | m_269862bba_9142334_16645812 | −5.6 | 1.1 |
| TTA005 | m_269862bba_8696858_14761790 | −5.5 | 1.2 |
| TTA020 | m_269862bba_9631398_15157990 | −4.6 | 1.5 |
| TTA036 | m_270004bba_9142336_8334522 | −4.2 | 1.3 |
| TTA019 | m_269862bba_8258822_9779938 | −4.0 | 1.5 |
| TTA031 | m_269862bba_13913074_13817824 | −3.6 | 0.9 |
| TTA024 | m_44daa_13913046_202575 | −3.2 | 1.4 |
| TTA022 | m_269862bba_17312426_9779938 | −2.5 | 1.6 |
| TTA017 | m_269862bba_13913074_13817824 | −2.0 | 1.5 |
| TTA029 | m_270004bba_13913076_9736040 | −2.0 | 1.9 |
| TTA023 | m_22bcc_20995888_2989428 | −1.9 | 1.4 |
| TTA032 | m_269862bba_13913074_7201654 | −1.8 | 0.9 |
| TTA010 | m_269862bba_8949394_9779938 | −1.4 | 1.2 |
| TTA007 | m_269862bba_10740898_9779938 | −0.8 | 1.6 |
| TTA034 | m_269862bba_13913074_16603894 | −0.8 | 0.9 |
| TTA014 | m_44daa_13913046_202575 | −0.7 | 1.0 |
| TTA016 | m_269862bba_8696858_14761790 | −0.4 | 1.7 |
| TTA003 | m_270004bba_13913076_12420056 | −0.3 | 1.4 |
| TTA001 | psilocin | 0.0 | 0.0 |
| TTA002 | m_269862bba_7197908_9779938 | 0.3 | 1.5 |
| TTA028 | m_269862bba_9142334_16603894 | 0.5 | 1.2 |
| TTA008 | m_269862bba_7192560_9779938 | 0.8 | 0.5 |
| TTA025 | m_269862bba_8350636_14761790 | 2.6 | 0.9 |
| TTA021 | m_273610aaa_13913088_11516246 | 3.1 | 1.9 |
| TTA018 | m_269862bba_8258822_9779938 | 4.6 | 1.0 |
| TTA040 | m_2230bbc_6465434_13206110_modified | 5.5 | 1.3 |
| TTA026 | m_270004bea_8297176_8157306 | 5.9 | 1.1 |
| TTA027 | m_269862bba_7194812_9779938 | 7.5 | 2.0 |
| TTA004 | m_22bbb_10548322_2440422 | 9.2 | 1.6 |

G—Calculated FEP binding affinities relative to psilocin.
* - Unconverged

It will be understood that reference to specific ligands in this disclosure will also include reference to their pharmaceutically acceptable salts (e.g., chlorides, acetates, formates, etc.), and their diastereomers.

### Example 2

The following are characterization data for the compounds in Table 1.

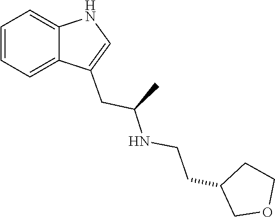

### TTA005

| | |
|---|---|
| Name | [1-(1H-indol-3-yl)propan-2-yl][2-(oxolan-3-yl)ethyl]amine |
| SMILES | CC(CC1═CNC═2C═CC═CC12)NCCC3CCOC3 |
| Stereochem. | Diastereomeric mixture |
| Formula | C17H24N2O |
| MW | 272.3853 |
| Purity | 93 |
| Ligand # | TTA005 |

¹H NMR (301 MHZ, MeOD) δ 7.54 (d, J=8.6 Hz, 1H), 7.34 (d, J=7.6 Hz, 1H), 7.18-6.85 (m, 3H), 3.81-3.48 (m, 3H), 3.17 (td, J=7.6, 3.3 Hz, 1H), 2.98 (p, J=6.3 Hz, 1H), 2.80 (d, J=6.8 Hz, 2H), 2.69-2.53 (m, 1H), 2.54-2.33 (m, 1H), 2.02-1.64 (m, 2H), 1.54-1.17 (m, 3H), 1.10 (dt, J=6.3, 1.4 Hz, 3H). ¹³C NMR (76 MHZ, MeOD) & 20.1, 33.0, 33.1, 33.8, 38.3, 46.9, 46.9, 54.8, 54.9, 68.7, 74.0, 74.0, 112.4, 113.0, 119.4, 119.7, 122.5, 124.2, 128.8, 138.3. HRMS: calcd for C₁₇H₂₄N₂O, [M+H]⁺=273.1961. found 273.1963.

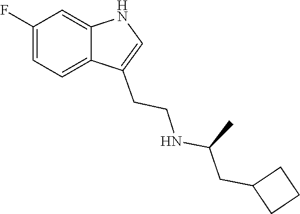 [SMILES: C[C@@H](CC1CCC1)NCCc1c[nH]c2cc(F)ccc12]

### TTA009

| | |
|---|---|
| Name | (1-cyclobutylpropan-2-yl)[2-(6-fluoro-1H-indol- |
| | 3-yl)ethyl]amine |
| SMILES | CC(CC1CCC1)NCCC2═CNC═3C═C(F)C═CC23 |
| Stereochem. | Racemic or presumed racemic or meso |
| Formula | C17H23FN2 |
| MW | 274.3763 |
| Purity | 100 |
| Ligand # | TTA009 |

¹H NMR (301 MHZ, MeOD) δ 7.61-7.33 (m, 1H), 7.05 (d, J=11.4 Hz, 2H), 6.90-6.61 (m, 1H), 3.05-2.78 (m, 4H), 2.72-2.51 (m, 1H), 2.16 (h, J=7.7 Hz, 1H), 1.98-1.63 (m, 4H), 1.63-1.42 (m, 3H), 1.35 (dt, J=13.8, 7.3 Hz, 1H), 1.01 (dd, J=6.4, 1.2 Hz, 3H). ¹³C NMR (76 MHz, MeOD) δ 170.35, 161.23 (d, J=235.5 Hz), 138.21 (d, J=13.3 Hz), 125.27, 124.20 (d, J=3.3 Hz), 120.07 (d, J=10.2 Hz), 113.02, 108.18 (d, J=24.9 Hz), 98.28 (d, J=26.0 Hz), 53.24, 47.65, 44.20, 34.37, 29.79, 29.45, 25.51, 19.49, 19.34. ¹⁹F NMR (283 MHZ, MeOD) δ−121.2. HRMS calcd for C₁₇H₂₃FN₂, [M+H]⁺=275.1918. found 275.1921.

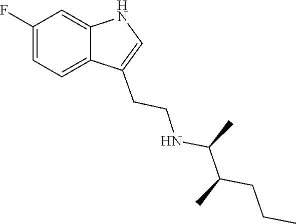 [SMILES: CCC[C@@H](C)[C@H](C)NCCc1c[nH]c2cc(F)ccc12]

### TTA011

| | |
|---|---|
| Name | [2-(6-fluoro-1H-indol-3-yl)ethyl](3-methylhexan-2-yl)amine |
| SMILES | CCCC(C)C(C)NCCC1═CNC═2C═C(F)C═CC12 |
| Stereochem. | Diastereomeric mixture |
| Formula | C17H25FN2 |
| MW | 276.3922 |
| Purity | 100 |
| Ligand # | TTA011 |

¹H NMR (301 MHZ, MeOD) δ 7.48 (dd, J=8.7, 5.3 Hz, 1H), 7.21-6.96 (m, 2H), 6.78 (ddd, J=9.8, 8.7, 2.3 Hz, 1H), 3.11-2.70 (m, 4H), 2.59-2.31 (m, 1H), 1.62-1.36 (m, 1H), 1.37-0.84 (m, 7H), 0.84-0.66 (m, 6H). ¹³C NMR (76 MHZ, MeOD) δ 161.23 (d, J=235.2 Hz), 138.23 (d, J=15.1 Hz), 125.35, 124.09 (t, J=4.2 Hz), 120.13 (d, J=10.4 Hz), 113.60, 108.10 (d, J=24.9 Hz), 98.24 (d, J=25.9 Hz), 58.67, 58.02, 37.97, 37.38, 34.92, 25.91, 21.50, 21.26, 16.31, 16.14, 14.99, 14.58, 14.13. ¹⁹F NMR (283 MHZ, MeOD) δ−124.39 (d, J=3.1 Hz). HRMS calcd for C₁₇H₂₅FN₂, [M+H]⁺=277.2075. found 277.2077.

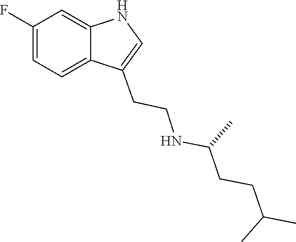 [SMILES: CC(C)CC[C@@H](C)NCCc1c[nH]c2cc(F)ccc12]

### TTA012

| | |
|---|---|
| Name | [2-(6-fluoro-1H-indol-3-yl)ethyl](5-methylhexan-2-yl)amine |
| SMILES | CC(C)CCC(C)NCCC1═CNC═2C═C(F)C═CC12 |
| Stereochem. | Racemic or presumed racemic or meso |
| Formula | C17H25FN2 |
| MW | 276.3922 |
| Purity | 100 |
| Ligand # | TTA012 |

¹H NMR (301 MHZ, MeOD) δ 7.48 (dd, J=8.7, 5.3 Hz, 1H), 7.14-6.96 (m, 2H), 6.78 (ddd, J=9.8, 8.7, 2.4 Hz, 1H), 3.01-2.72 (m, 4H), 2.66-2.43 (m, 1H), 1.51-1.13 (m, 3H), 1.10-0.91 (m, 5H), 0.80 (dd, J=6.6, 4.0 Hz, 6H). ¹³C NMR (76 MHZ, MeOD) δ 161.22 (d, J=235.0 Hz), 138.21 (d, J=12.4 Hz), 125.36, 124.06 (d, J=3.4 Hz), 120.10 (d, J=10.3 Hz), 113.54, 108.12 (d, J=24.8 Hz), 98.25 (d, J=25.9 Hz), 54.44, 47.88, 36.07, 35.04, 29.22, 25.93, 22.93, 22.84, 19.77. ¹⁹F NMR (283 MHz, MeOD) δ−124.4. HRMS calcd for C₁₇H₂₅FN₂, [M+H]⁺=277.2075. found 277.2076.

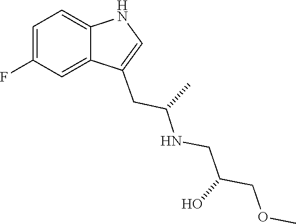 [SMILES: COC[C@H](O)CN[C@@H](C)Cc1c[nH]c2ccc(F)cc12]

### TTA015

| | |
|---|---|
| Name | [1-(5-fluoro-1H-indol-3-yl)propan-2-yl][(2R)-2-hydroxy-3- |
| | methoxypropyl]amine |
| SMILES | COC[C@H](O)CNC(C)CC1═CNC═2C═CC(F)═CC12 |
| Stereochem. | Diastereomeric mixture |
| Formula | C15H21FN2O2 |
| MW | 280.3378 |
| Purity | 99 |
| Ligand # | TTA015 |

¹H NMR (301 MHZ, MeOD) δ 8.57 (s, 1H), 7.28 (dd, J=8.8, 4.5 Hz, 1H), 7.21 (dd, J=9.9, 2.5 Hz, 1H), 7.14 (s, 1H), 6.85 (td, J=9.2, 2.5 Hz, 1H), 3.79 (ddt, J=10.2, 8.8, 4.1 Hz, 1H), 3.36-3.18 (m, 5H), 3.06-2.94 (m, 1H), 2.88-2.70 (m, 3H), 2.46 (dd, J=12.0, 8.8 Hz, 1H), 1.13 (d, J=6.2 Hz, 3H). ¹³C NMR (76 MHZ, MeOD) δ 19.6, 19.8, 33.2, 33.4, 50.7, 51.0, 54.9, 55.1, 59.3, 69.4, 69.8, 76.6, 103.9, 104.2, 110.3, 110.3, 110.6, 110.7, 112.8, 112.8, 113.0, 113.1, 126.3, 129.1, 129.2, 134.8, 134.8, 157.4, 160.4, 170.3. ¹⁹F NMR (283 MHz, MeOD) δ−127.7, −127.7. HRMS calcd for C₁₅H₂₁FN₂O₂, [M+H]⁺=281.1660. found 281.1661.

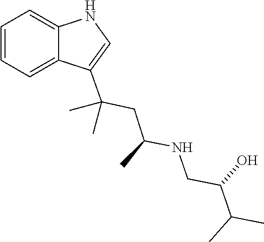 [SMILES: CC(C)[C@@H](O)CN[C@@H](C)CC(C)(C)c1c[nH]c2ccccc12]

### TTA019

| | |
|---|---|
| Name | 1-{[4-(1H-indol-3-yl)-4-methylpentan-2- |
| | yl]amino}-3-methylbutan-2-ol |
| SMILES | CC(C)C(O)CNC(C)CC(C)(C)C1═CNC═2C═CC═CC12 |
| Stereochem. | Diastereomeric mixture |
| Formula | C19H30N2O |
| MW | 302.4543 |
| Purity | 100 |
| Ligand # | TTA019 |

¹H NMR (301 MHZ, MeOD) δ 7.79-7.68 (m, 1H), 7.34 (ddd, J=7.2, 2.4, 1.2 Hz, 1H), 7.12-6.92 (m, 3H), 2.93 (tt, J=10.0, 5.7 Hz, 1H), 2.68-2.47 (m, 1H), 2.38 (dd, J=12.0, 3.1 Hz, OH), 2.28-1.91 (m, 2H), 1.89-1.67 (m, 1H), 1.53-1.35 (m, 6H), 1.20 (dq, J=13.1, 6.7 Hz, OH), 1.08-0.84 (m, 4H), 0.82-0.57 (m, 6H). ¹³C NMR (76 MHz, MeOD) δ 17.2, 17.8, 19.2, 19.2, 22.0, 22.3, 29.3, 29.5, 30.9, 31.1, 32.8, 33.4, 35.4, 51.3, 51.4, 52.2, 52.4, 75.4, 75.5, 112.7, 119.4, 119.5, 121.9, 122.1, 122.1, 122.3, 122.4, 123.5, 126.9, 139.1. HRMS: calcd for C₁₉H₃₀N₂O, [M+H]⁺=303.2431. found 303.2437.

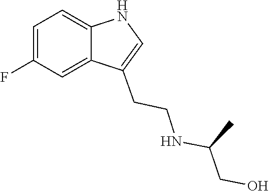 [SMILES: C[C@@H](CO)NCCc1c[nH]c2ccc(F)cc12]

### TTA020

| | |
|---|---|
| Name | 2-{[2-(5-fluoro-1H-indol-3-yl)ethyl]amino}propan-1-ol |
| SMILES | CC(CO)NCCC1═CNC═2C═CC(F)═CC12 |
| Stereochem. | Racemic or presumed racemic or meso |
| Formula | C13H17FN2O |
| MW | 236.2853 |
| Purity | 91 |
| Ligand # | TTA020 |

¹H NMR (301 MHZ, MeOD) δ 7.52 (s, 1H), 7.16-6.92 (m, 2H), 6.78 (ddd, J=9.7, 8.6, 2.4 Hz, 1H), 3.47 (dd, J=10.8, 4.7 Hz, 1H), 3.41-3.25 (m, 1H), 3.05-2.65 (m, 5H), 1.00 (d, J=6.4 Hz, 3H). ¹³C NMR (76 MHZ, MeOD) δ 161.17 (d, J=235.2 Hz), 138.14 (d, J=12.4 Hz), 125.40, 123.90, 120.10 (d, J=10.2 Hz), 113.71, 108.01 (d, J=24.9 Hz), 98.14 (d, J=26.0 Hz), 66.71, 55.73, 48.14, 26.21, 16.54. ¹⁹F NMR (283 MHZ, MeOD) δ−121.5. HRMS calcd for C₁₃H₁₇FN₂O, [M+H]⁺=237.1398. found 237.1398.

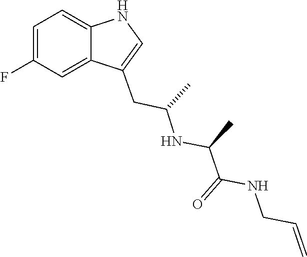 [SMILES: C=CCNC(=O)[C@H](C)N[C@@H](C)Cc1c[nH]c2ccc(F)cc12]

### TTA024

| | |
|---|---|
| Name | 2-{[1-(5-fluoro-1H-indol-3-yl)propan-2-yl]amino}-N- |
| | (prop-2-en-1-yl)propanamide |
| SMILES | CC(CC1═CNC═2C═CC(F)═CC12)NC(C)C(═O)NCC═C |
| Stereochem. | Diastereomeric mixture |
| Formula | C17H22FN3O |
| MW | 303.3745 |
| Purity | 100 |
| Ligand # | TTA024 |

¹H NMR (301 MHZ, MeOD) δ 7.29 (dd, J=8.8, 4.4 Hz, 1H), 7.20 (ddd, J=9.9, 4.1, 2.5 Hz, 1H), 7.13 (d, J=9.7 Hz, 1H), 6.86 (td, J=9.1, 2.5 Hz, 1H), 5.96-5.50 (m, 1H), 5.26-5.06 (m, 1H), 5.04-4.95 (m, 1H), 3.83 (ddt, J=5.4, 3.4, 1.7 Hz, 1H), 3.62 (ddt, J=15.7, 5.3, 1.7 Hz, 1H), 3.43 (q, J=6.8 Hz, 1H), 3.33 (qt, J=6.8, 2.6 Hz, 1H), 3.00-2.87 (m, 1H), 2.85-2.56 (m, 2H), 1.20 (t, J=6.7 Hz, 3H), 1.16-0.99 (m, 3H). ¹³C NMR (76 MHZ, MeOD) δ 177.89 (d, J=26.4 Hz), 160.39 (d, J=2.8 Hz), 157.32 (d, J=2.7 Hz), 135.20 (d, J=14.9 Hz), 134.71 (d, J=2.3 Hz), 129.23 (dd, J=9.4, 5.8 Hz), 126.19 (d, J=2.8 Hz), 116.24 (d, J=33.7 Hz), 113.81 (d, J=4.9 Hz), 113.33 (d, J=4.8 Hz), 113.03 (d, J=9.6 Hz), 110.39 (d, J=26.5 Hz), 104.13 (dd, J=23.4, 12.9 Hz), 56.46, 56.10, 53.88, 53.58, 42.49, 42.10, 34.69, 33.05, 21.19, 20.14, 20.07, 19.79. ¹⁹F NMR (283 MHZ, MeOD) δ−127.7, −127.7. HRMS calcd for C₁₇H₂₂FN₃O, [M+H]⁺=304.1820. found 304.1824.

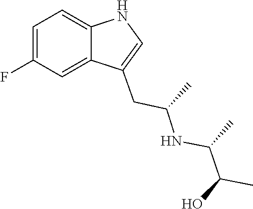 [SMILES: C[C@@H](Cc1c[nH]c2ccc(F)cc12)N[C@H](C)[C@@H](C)O]

### TTA031

| | |
|---|---|
| Name | 3-{[1-(5-fluoro-1H-indol-3-yl)propan-2- |
| | yl]amino}butan-2-ol |
| SMILES | CC(O)C(C)NC(C)CC1═CNC═2C═CC(F)═CC12 |
| Stereochem. | Diastereomeric mixture |
| Formula | C15H21FN2O |
| MW | 264.3384 |
| Purity | 91 |
| Ligand # | TTA031 |

¹H NMR (301 MHZ, MeOD) δ 7.30 (dd, J=8.8, 4.5 Hz, 1H), 7.22 (dd, J=9.9, 2.5 Hz, 1H), 7.15 (s, 1H), 6.86 (td, J=9.1, 2.5 Hz, 1H), 3.74 (qd, J=6.5, 3.4 Hz, 1H), 3.13 (q, J=6.5 Hz, 1H), 2.83 (dd, J=14.2, 6.6 Hz, 1H), 2.78-2.60 (m, 2H), 1.09 (d, J=6.3 Hz, 3H), 1.04 (dd, J=6.6, 1.3 Hz, 6H). ¹³C NMR (76 MHz, MeOD) δ 158.84 (d, J=232.0 Hz), 134.72, 129.35 (d, J=9.6 Hz), 126.13, 113.43 (d, J=4.7 Hz), 112.98 (d, J=9.6 Hz), 110.36 (d, J=26.5 Hz), 104.02 (d, J=23.4 Hz), 69.11, 56.30, 52.09, 33.74, 20.81, 19.29, 15.27. ¹⁹F NMR (283 MHZ, MeOD) δ−127.77. HRMS calcd for C₁₅H₂₁FN₂O, [M+H]⁺=265.1711. found 265.1711.

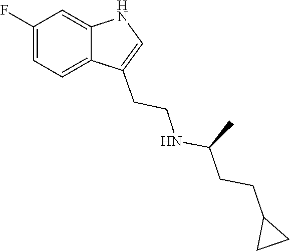 [SMILES: C[C@@H](CCC1CC1)NCCc1c[nH]c2cc(F)ccc12]

### TTA033

| | |
|---|---|
| Name | (4-cyclopropylbutan-2-yl)[2-(6-fluoro-1H-indol- |
| | 3-yl)ethyl]amine |
| SMILES | CC(CCC1CC1)NCCC2═CNC═3C═C(F)C═CC23 |
| Stereochem. | Racemic or presumed racemic or meso |
| Formula | C17H23FN2 |
| MW | 274.3763 |
| Purity | 100 |
| Ligand # | TTA033 |

¹H NMR (301 MHz, MeOD) δ 8.59 (s, 1H), 7.49 (dd, J=8.7, 5.3 Hz, 1H), 7.12-6.98 (m, 2H), 6.80 (td, J=9.7, 2.4 Hz, 1H), 3.12-2.86 (m, 4H), 2.78 (h, J=6.4 Hz, 1H), 1.61 (dtd, J=13.4, 8.0, 5.2 Hz, 1H), 1.50-1.29 (m, 1H), 1.08 (dd, J=7.0, 3.6 Hz, 5H), 0.70-0.47 (m, 1H), 0.35 (d, J=7.5 Hz, 2H), 0.02-−0.18 (m, 2H). ¹³C NMR (76 MHZ, MeOD) δ 170.34, 161.24 (d, J=235.1 Hz), 138.20 (d, J=12.5 Hz), 125.25, 124.21 (d, J=3.4 Hz), 120.02 (d, J=10.3 Hz), 112.90, 108.21 (d, J=24.9 Hz), 98.30 (d, J=26.0 Hz), 54.29, 47.54, 36.51, 31.92, 25.36, 18.98, 11.53, 5.01, 4.84. ¹⁹F NMR (283 MHZ, MeOD) δ−127.58. HRMS calcd for C₁₇H₂₃FN₂, [M+H]⁺=275.1918. found 275.1924.

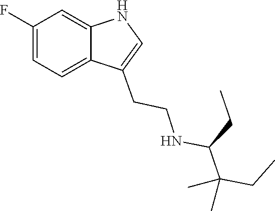 [SMILES: CC[C@H](NCCc1c[nH]c2cc(F)ccc12)C(C)(C)CC]

### TTA035

| | |
|---|---|
| Name | (4,4-dimethylhexan-3-yl)[2-(6-fluoro-1H-indol- |
| | 3-yl)ethyl]amine |
| SMILES | CCC(NCCC1═CNC═2C═C(F)C═CC12)C(C)(C)CC |
| Stereochem. | Racemic or presumed racemic or meso |
| Formula | C18H27FN2 |
| MW | 290.4188 |
| Purity | 100 |
| Ligand # | TTA035 |

¹H NMR (301 MHZ, MeOD) δ 7.50 (dd, J=8.7, 5.3 Hz, 1H), 7.08-7.01 (m, 2H), 6.79 (ddd, J=9.7, 8.7, 2.4 Hz, 1H), 3.10-2.71 (m, 4H), 1.92 (dd, J=8.2, 3.2 Hz, 1H), 1.56 (dqd, J=14.9, 7.6, 3.2 Hz, 1H), 1.25-1.00 (m, 3H), 0.89 (t, J=7.5 Hz, 3H), 0.68 (d, J=2.9 Hz, 6H), 0.61 (t, J=7.5 Hz, 3H). ¹³C NMR (76 MHZ, MeOD) δ 161.21 (d, J=234.9 Hz), 138.24 (d, J=12.5 Hz), 125.44, 124.19 (d, J=3.5 Hz), 120.23 (d, J=10.2 Hz), 113.83, 108.05 (d, J=24.9 Hz), 98.18 (d, J=25.7 Hz), 69.57, 52.75, 38.57, 32.87, 25.77 (d, J=103.6 Hz), 23.88, 23.73, 13.67, 8.32. ¹⁹F NMR (283 MHz, MeOD) δ−124.4. HRMS calcd for C₁₈H₂₇FN₂, [M+H]⁺=291.2231. found 291.1234.

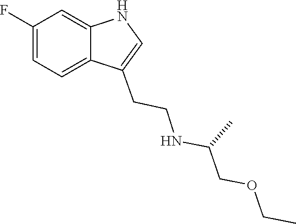 [SMILES: CCOC[C@@H](C)NCCc1c[nH]c2cc(F)ccc12]

### TTA036

| | |
|---|---|
| Name | (1-ethoxypropan-2-yl)[2-(6-fluoro-1H-indol- |
| | 3-yl)ethyl]amine |
| SMILES | CCOCC(C)NCCC1═CNC═2C═C(F)C═CC12 |
| Stereochem. | Racemic or presumed racemic or meso |
| Formula | C15H21FN2O |
| MW | 264.3384 |
| Purity | 98 |
| Ligand # | TTA036 |

¹H NMR (301 MHZ, MeOD) δ 8.57 (s, 1H), 7.47 (dd, J=8.7, 5.3 Hz, 1H), 7.06 (s, 1H), 7.02 (dd, J=10.0, 2.4 Hz, 1H), 6.77 (ddd, J=9.6, 8.7, 2.4 Hz, 1H), 3.43-3.23 (m, 3H), 3.21 (dd, J=9.8, 7.6 Hz, 1H), 3.09-2.80 (m, 3H), 1.11-0.93 (m, 6H). ¹³C NMR (76 MHz, MeOD) δ 170.33, 161.25 (d, J=235.0 Hz), 138.23 (d, J=12.5 Hz), 125.31, 124.21 (d, J=3.4 Hz), 120.06 (d, J=10.2 Hz), 113.02, 108.13 (d, J=24.9 Hz), 98.23 (d, J=25.9 Hz), 74.33, 67.45, 53.81, 47.53, 25.53, 16.14, 15.25. ¹⁹F NMR (283 MHz, MeOD) δ−124.44 (td, J=10.1, 5.5 Hz). HRMS calcd for C₁₅H₂₁FN₂O, [M+H]⁺=265.1711. found 265.1713.

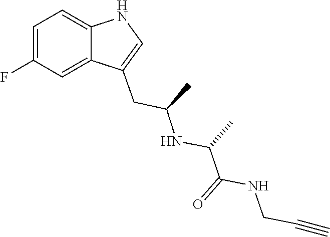 [SMILES: C#CCNC(=O)[C@@H](C)N[C@H](C)Cc1c[nH]c2ccc(F)cc12]

### TTA037

| | |
|---|---|
| Name | 2-{[1-(5-fluoro-1H-indol-3-yl)propan-2-yl]amino}-N- |
| | (prop-2-yn-1-yl)propanamide |
| SMILES | CC(CC1═CNC═2C═CC(F)═CC12)NC(C)C(═O)NCC#C |
| Stereochem. | Diastereomeric mixture |
| Formula | C17H20FN3O |
| MW | 301.3586 |
| Purity | 98 |
| Ligand # | TTA037 |

¹H NMR (301 MHZ, MeOD) δ 7.37-7.24 (m, 1H), 7.19 (dt, J=9.9, 2.5 Hz, 1H), 7.13 (d, J=10.9 Hz, 1H), 6.92-6.80 (m, 1H), 4.08-3.90 (m, 1H), 3.85-3.51 (m, 1H), 3.46-3.32 (m, 1H), 3.00-2.60 (m, 3H), 2.60-2.45 (m, 1H), 1.18 (dd, J=7.9, 6.8 Hz, 3H), 1.12-0.95 (m, 3H). ¹³C NMR (76 MHZ, MeOD) δ 177.75 (d, J=27.6 Hz), 158.84 (d, J=231.9 Hz), 134.69, 126.21, 113.03 (dd, J=9.7, 2.8 Hz), 110.40 (d, J=26.5 Hz), 104.27 (d, J=9.4 Hz), 103.96 (d, J=9.4 Hz), 80.43, 80.36, 72.26, 71.99, 56.37, 55.91, 53.65, 53.56, 34.57, 33.04, 29.27, 28.97, 21.11, 19.93, 19.81, 19.52. ¹⁹F NMR (283 MHz, MeOD) δ−131.00 (d, J=6.7 Hz). HRMS calcd for C₁₇H₂₀FN₃O, [M+H]⁺=302.1663. found 302.1664.

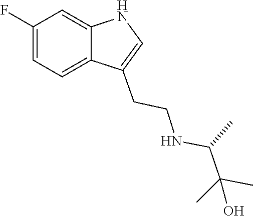 [SMILES: C[C@@H](NCCc1c[nH]c2cc(F)ccc12)C(C)(C)O]

### TTA038

| | |
|---|---|
| Name | 3-{[2-(6-fluoro-1H-indol-3-yl)ethyl]amino}- |
| | 2-methylbutan-2-ol |
| SMILES | CC(NCCC1═CNC═2C═C(F)C═CC12)C(C)(C)O |
| Stereochem. | Racemic or presumed racemic or meso |
| Formula | C15H21FN2O |
| MW | 264.3384 |
| Purity | 100 |
| Ligand # | TTA038 |

¹H NMR (301 MHz, MeOD) δ 7.49 (dd, J=8.7, 5.3 Hz, 1H), 7.07 (d, J=1.1 Hz, 1H), 7.02 (dd, J=10.0, 2.3 Hz, 1H), 6.84-6.73 (m, 1H), 3.13-2.81 (m, 3H), 2.72 (dt, J=9.6, 6.9 Hz, 1H), 2.47 (q, J=6.6 Hz, 1H), 1.13 (s, 3H), 1.06 (s, 3H), 1.02 (d, J=6.6 Hz, 3H). ¹³C NMR (76 MHZ, MeOD) δ 161.19 (d, J=234.8 Hz), 138.15 (d, J=12.7 Hz), 125.50, 123.91 (d, J=3.5 Hz), 120.15 (d, J=10.2 Hz), 113.99, 107.97 (d, J=24.8 Hz), 98.11 (d, J=25.9 Hz). 19F NMR (283 MHz, MeOD) δ−124.7. HRMS calcd for C₁₅H₂₁FN₂O, [M+H]⁺=265.1711. found 265.1713.

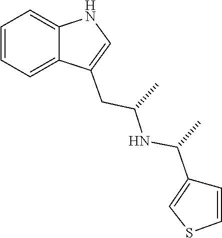 [SMILES: C[C@@H](Cc1c[nH]c2ccccc12)N[C@H](C)c1ccsc1]

### TTA039

| | |
|---|---|
| Name | [1-(1H-indol-3-yl)propan-2-yl][1-(thiophen- |
| | 3-yl)ethyl]amine |
| SMILES | CC(CC1═CNC═2C═CC═CC12)NC(C)C═3C═CSC3 |
| Stereochem. | Diastereomeric mixture |
| Formula | C17H20N2S |
| MW | 284.4191 |
| Purity | 100 |
| Ligand # | TTA039 |

¹H NMR (301 MHZ, MeOD) δ 7.39-7.24 (m, 3H), 7.18-7.02 (m, 3H), 7.01-6.50 (m, 2H), 4.08 (q, J=6.6 Hz, 1H), 3.07-2.85 (m, 1H), 2.85-2.67 (m, 1H), 2.60 (dt, J=13.3, 6.2 Hz, 1H), 1.37-1.20 (m, 3H), 1.06 (d, J=5.8 Hz, 1H), 0.94 (d, J=6.1 Hz, 2H). ¹³C NMR (76 MHz, MeOD) & 18.4, 19.7, 22.0, 22.5, 31.4, 32.9, 49.9, 50.2, 50.5, 50.5, 110.8, 111.5, 111.9, 118.1, 118.2, 118.2, 118.3, 120.0, 120.4, 120.9, 121.1, 122.6, 122.7, 124.9, 125.3, 125.5, 125.7, 127.4, 127.6, 136.8, 136.9, 145.6, 146.4. HRMS: calcd for C₁₇H₂₀N₂S, [M+H]⁺=285.1420. found 285.1424.

### Example 3: Structure-Based Virtual Screening

From the initial ranked dataset, the 500 best-ranked compounds were analyzed, and ˜90 were selected for MDs. Briefly, the conformations of top-ranked compounds were consistent with the interaction profile of co-crystalized agonists, i.e., psilocin, LSD, 25CN-NBOH. Based on the SBVS, a huge number of compounds generated a reliable binding pose deep inside the binding pocket at the bottom hydrophobic cleft, surrounded by highly conserved aromatic and hydrophobic residues (including Ile163 and Phe332 in the PIF motif and the toggle switch Trp336) and close to the side-extended cavity located between transmembrane helix 4 and 5. Among the top hits, all compounds revealed conserved H-bond interaction with Asp155 and hydrophobic interactions with Phe339, Trp336 (toggle switch), and Leu229.

We identified 21 ligands out of 82 initial hits (numbered TTM001, TTM002, TTM003, TTM004, TTM005, TTM006, TTM009, TTM013, TTM017, TTM019, TTM024, TTM028, TTM034, TTM038, TTM041, TTM045, TTM048, TTM057, TTM059, TTM068, TTM082). Their structures are shown in Table 3.

The binding energy profiles of the ligands in Table 3 compared to psilocin are shown in Table 4.

TABLE 3
Structures

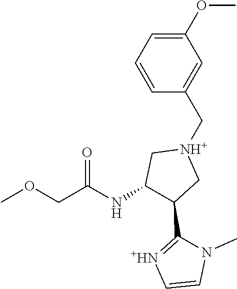

TTM001

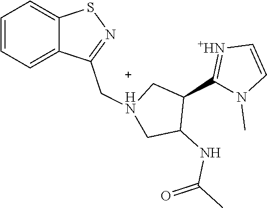

TTM002

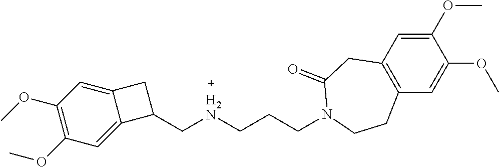

TTM003

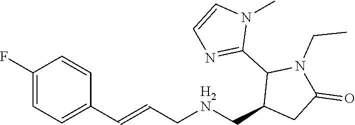

TTM004

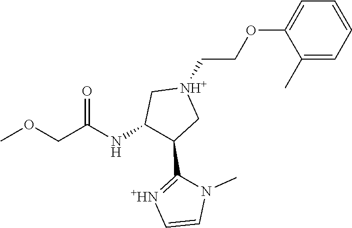 [SMILES: COCC(=O)N[C@@H]1C[N@H+](CCOc2ccccc2C)C[C@H]1c1[nH+]ccn1C]

TTM005

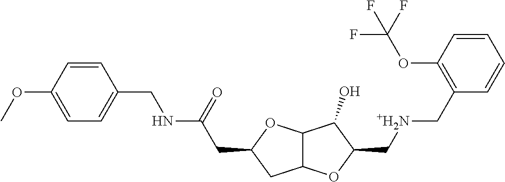 [SMILES: COc1ccc(CNC(=O)C[C@@H]2CC3O[C@H](C[NH2+]Cc4ccccc4OC(F)(F)F)[C@@H](O)C3O2)cc1]

TTM006

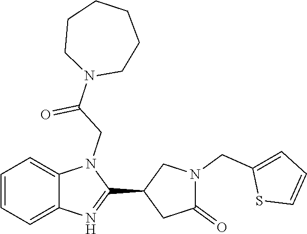 [SMILES: O=C(Cn1c([C@@H]2CC(=O)N(Cc3cccs3)C2)nc2ccccc21)N1CCCCCC1]

TTM009

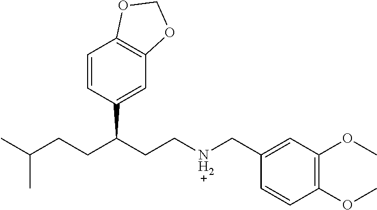 [SMILES: COc1ccc(C[NH2+]CC[C@@H](CCC(C)C)c2ccc3c(c2)OCO3)cc1OC]

TTM013

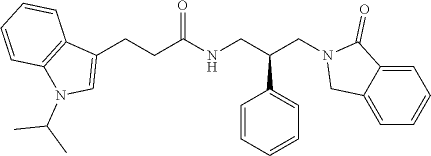 [SMILES: CC(C)n1cc(CCC(=O)NC[C@@H](CN2Cc3ccccc3C2=O)c2ccccc2)c2ccccc21]

TTM017

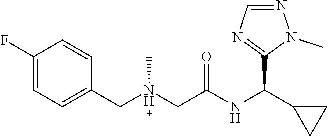 [SMILES: Cn1ncnc1[C@H](NC(=O)C[N@@H+](C)Cc1ccc(F)cc1)C1CC1]

TTM019

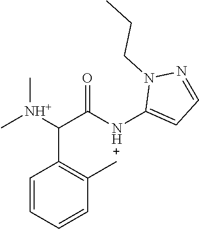 [SMILES: CCCn1nccc1[NH2+]C(=O)C(c1ccccc1C)[NH+](C)C]

TTM024

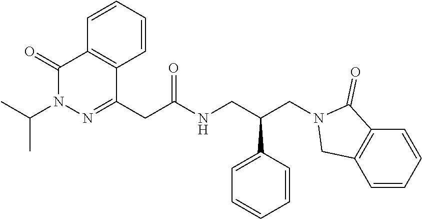 [SMILES: CC(C)n1nc(CC(=O)NC[C@@H](CN2Cc3ccccc3C2=O)c2ccccc2)c2ccccc2c1=O]

TTM028

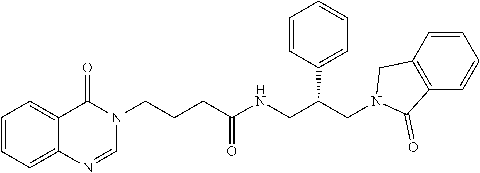 [SMILES: O=C(CCCn1cnc2ccccc2c1=O)NC[C@@H](CN1Cc2ccccc2C1=O)c1ccccc1]

TTM034

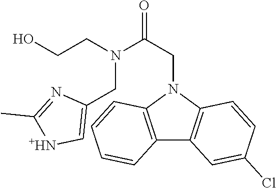 [SMILES: CC1=NC(CN(CCO)C(=O)Cn2c3ccccc3c3cc(Cl)ccc32)=C[NH2+]1]

TTM038

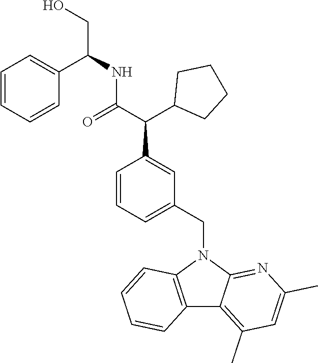 [SMILES: Cc1cc(C)c2c3ccccc3n(Cc3cccc([C@@H](C(=O)N[C@H](CO)c4ccccc4)C4CCCC4)c3)c2n1]

TTM041

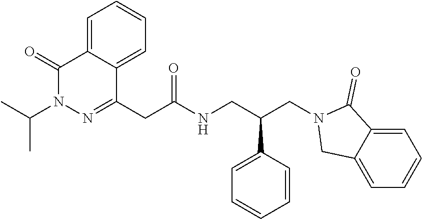 [SMILES: CC(C)n1nc(CC(=O)NC[C@@H](CN2Cc3ccccc3C2=O)c2ccccc2)c2ccccc2c1=O]

TTM057

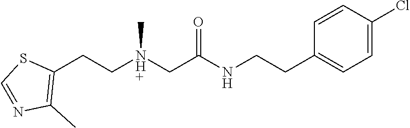 [SMILES: Cc1ncsc1CC[N@@H+](C)CC(=O)NCCc1ccc(Cl)cc1]

TTM059

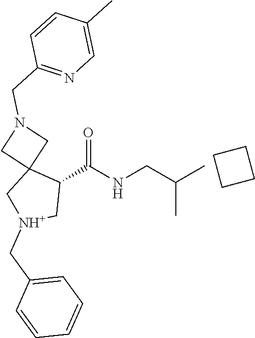

TTM068

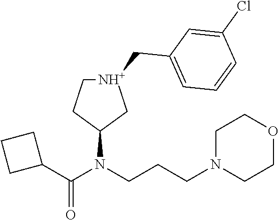 [SMILES: O=C(C1CCC1)N(CCCN1CCOCC1)[C@H]1CC[N@@H+](Cc2cccc(Cl)c2)C1]

TTM082

TABLE 4
Docking score and Prime MMGBSA score and QikProp properties of best hits

| Ligand# | XP Score | A | B | C | D | E | F | G |
|---|---|---|---|---|---|---|---|---|
| Psilocin | −8.8 | −63.1 | −4.9 | | 637.945 | 0.20 | 336.685 | −28.15 |
| TTM001 | −13.719 | −62.36 | −4.984 | −2.149 | 650.514 | 0.133 | 485.293 | −44.46 |
| TTM002 | −13.323 | −76.13 | −5.159 | −2.843 | 351.967 | 0.19 | 526.888 | −34.74 |
| TTM003 | −13.253 | −79.59 | −5.401 | −4.033 | 448.251 | −0.238 | 435.293 | −44.44 |
| TTM004 | −12.86 | −58.34 | −4.73 | −3.142 | 355.997 | 0.165 | 668.928 | −46.5 |
| TTM005 | −12.851 | −77.21 | −5.265 | −2.425 | 659.351 | 0.064 | 487.979 | −39.51 |
| TTM006 | −12.82 | −60.35 | −4.672 | −4.669 | 334.207 | −0.083 | 1155.237 | −46.64 |
| TTM007 | −12.753 | −54.52 | −5.401 | −9.278 | 1308.087 | −0.516 | 2131.685 | −49.88 |
| TTM008 | −12.601 | −52.49 | −4.214 | −2.874 | 358.573 | 0.252 | 959.085 | −37.94 |
| TTM009 | −12.577 | −59.53 | −2.292 | −4.14 | 1070.171 | −0.165 | 1955.249 | −44.91 |
| TTM010 | −12.538 | −46.66 | −4.978 | −3.481 | 810.243 | 0.616 | 1909.76 | −38.81 |
| TTM011 | −12.53 | −67.46 | −5.144 | −4.735 | 340.54 | −0.877 | 671.642 | −56.75 |
| TTM012 | −12.447 | −53.77 | −4.392 | −3.414 | 529.278 | 0.648 | 3351.977 | −42.54 |
| TTM013 | −12.35 | −65.04 | −4.707 | −5.327 | 1620.492 | 0.134 | 922.217 | −44.69 |
| TTM014 | −12.337 | −54.45 | −4.33 | −1.951 | 306.173 | 0.051 | 451.475 | −33.63 |
| TTM015 | −12.266 | −47.24 | −4.882 | −7.721 | 1049.728 | −0.957 | 728.152 | −47 |
| TTM016 | −12.245 | −51.61 | −5.045 | −3.925 | 703.127 | 0.238 | 886.493 | −36.31 |
| TTM017 | −12.217 | −69.13 | −5.057 | −7.516 | 3405.304 | −0.358 | 2382.835 | −43.27 |
| TTM018 | −12.204 | −60.5 | −4.892 | −2.965 | 314.894 | 0.345 | 972.473 | −54.93 |
| TTM019 | −12.183 | −72.31 | −4.141 | −1.952 | 335.549 | 0.053 | 554.869 | −48.11 |
| TTM020 | −12.182 | −66.15 | −5.003 | −2.599 | 402.436 | 0.122 | 346.375 | −41.77 |
| TTM021 | −12.168 | −56.88 | −4.261 | −2.602 | 331.776 | 0.03 | 732.706 | −43.85 |
| TTM022 | −12.147 | −58.76 | −4.905 | −2.806 | 986.299 | 0.407 | 539.205 | −46.6 |
| TTM023 | −12.131 | −66.77 | −4.833 | −2.299 | 358.494 | 0.203 | 1138.998 | −39.86 |
| TTM024 | −12.096 | −63.39 | −5.288 | −2.834 | 862.665 | 0.255 | 680.506 | −50.56 |
| TTM025 | −12.059 | −60.93 | −4.926 | −2.49 | 333.645 | 0.043 | 339.878 | −45.27 |
| TTM026 | −12.058 | −44.63 | −4.797 | −2.444 | 438.84 | 0.27 | 683.789 | −51.47 |
| TTM027 | −12.052 | −69.51 | −5.331 | −4.147 | 342.555 | −0.978 | 479.384 | −42.97 |
| TTM028 | −12.043 | −58.01 | −5.114 | −7.057 | 608.218 | −1.076 | 409.584 | −36.51 |
| TTM029 | −12.017 | −67.66 | −5.295 | −9.181 | 744.674 | −0.584 | 6398.353 | −54.6 |
| TTM030 | −12.01 | −63.13 | −5.293 | −4.875 | 1529.082 | 0.152 | 866.119 | −45.64 |
| TTM031 | −11.996 | −56.35 | −4.46 | −2.661 | 936.497 | 0.345 | 1246.017 | −32.89 |
| TTM032 | −11.995 | −53.89 | −4.744 | −5.234 | 850.202 | −0.832 | 582.616 | −41.84 |
| TTM033 | −11.98 | −50.12 | −2.545 | −2.957 | 390.384 | −0.475 | 1082.189 | −31.56 |
| TTM034 | −11.964 | −71.3 | −5.447 | −5.944 | 491.346 | −1.228 | 328.465 | −35.55 |
| TTM035 | −11.963 | −57.69 | −5.462 | −5.944 | 483.25 | −1.237 | 323.203 | −33.76 |
| TTM036 | −11.952 | −68 | −5.452 | −6.07 | 916.667 | −0.581 | 1652.012 | −44.83 |
| TTM037 | −11.949 | −44.88 | −5.179 | −4.173 | 1303.512 | 0.569 | 1315.525 | −54.18 |
| TTM038 | −11.943 | −73.58 | −4.353 | −5.194 | 839.827 | −0.632 | 1460.11 | −48.95 |
| TTM039 | −11.91 | −58.48 | −4.323 | −5.522 | 1021.661 | −0.631 | 1040.824 | −48.58 |
| TTM040 | −11.883 | −60.51 | −5.443 | −3.147 | 477.38 | 0.212 | 416.85 | −38.63 |
| TTM041 | −11.848 | −68.6 | −3.801 | −8.713 | 2326.464 | −0.53 | 1395.377 | −34.34 |
| TTM042 | −11.838 | −52.46 | −5.348 | −4.561 | 799.342 | 0.663 | 2747.642 | −30.51 |
| TTM043 | −11.83 | −61.1 | −4.908 | −5.327 | 1609.473 | 0.128 | 915.441 | −30.44 |
| TTM044 | −11.821 | −63.4 | −4.638 | −3.678 | 401.815 | −0.951 | 749.071 | −38.45 |
| TTM045 | −11.82 | −85.24 | −5.279 | −4.493 | 306.688 | −0.088 | 452.788 | −32.43 |
| TTM046 | −11.811 | −52.25 | −3.981 | −3.26 | 979.44 | 0.557 | 1656.326 | −32.02 |
| TTM047 | −11.81 | −51.81 | −5.3 | −6.496 | 2615.938 | −0.111 | 10000 | −42.33 |
| TTM048 | −11.807 | −70.63 | −5.105 | −7.216 | 389.982 | −1.082 | 317.118 | −33.21 |
| TTM049 | −11.801 | −52.22 | −5.437 | −4.232 | 527.874 | 0.203 | 969.192 | −39.6 |
| TTM050 | −11.795 | −50.11 | −5.317 | −2.999 | 301.687 | 0.229 | 602.244 | −32.27 |
| TTM051 | −11.766 | −49.26 | −3.117 | −4.021 | 891.204 | 0.146 | 4013.872 | −47.57 |
| TTM052 | −11.762 | −48.97 | −4.622 | −7.158 | 886.422 | −0.663 | 671.365 | −41.25 |
| TTM053 | −11.738 | −60.94 | −4.973 | −4.654 | 983.534 | −0.628 | 485.911 | −58.17 |
| TTM054 | −11.732 | −56.87 | −4.051 | −4.193 | 1931.194 | −0.519 | 1066.962 | −54.4 |
| TTM055 | −11.731 | −50.54 | −3.656 | −4.821 | 372.674 | −0.725 | 482.757 | −34.09 |
| TTM056 | −11.72 | −44.75 | −5.137 | −3.853 | 402.729 | 0.07 | 332.029 | −27.85 |
| TTM057 | −11.705 | −75.07 | −5.114 | −7.057 | 608.22 | −1.076 | 409.586 | −23.83 |
| TTM058 | −11.686 | −59.63 | −5.313 | −5.069 | 600.878 | 0.328 | 630.046 | −29.93 |
| TTM059 | −11.672 | −75.23 | −5.373 | −2.946 | 342.22 | −0.001 | 1180.815 | −27.29 |
| TTM060 | −11.671 | −60.17 | −5.182 | −5.739 | 528.857 | −0.922 | 566.882 | −28.91 |
| TTM061 | −11.667 | −58.57 | −4.972 | −6.874 | 695.863 | −0.817 | 502.231 | −59.25 |
| TTM062 | −11.644 | −68.13 | −4.917 | −3.991 | 401.673 | −1.02 | 448.345 | −55.47 |
| TTM063 | −11.642 | −55.37 | −4.91 | −1.927 | 828.303 | 0.448 | 1422.369 | −23.6 |
| TTM064 | −11.635 | −55.39 | −4.763 | −4.658 | 1872.596 | −0.018 | 4283.32 | −51.21 |
| TTM065 | −11.632 | −52.94 | −4.72 | −2.471 | 616.595 | 0.113 | 503.593 | −24.84 |
| TTM066 | −11.631 | −60.68 | −4.539 | −4.585 | 962.764 | −0.345 | 2079.409 | −51.36 |
| TTM067 | −11.629 | −57.88 | −4.786 | −5.464 | 310.308 | −1.34 | 459.343 | −34.86 |
| TTM068 | −11.613 | −62.33 | −5.307 | −4.163 | 704.378 | 0.138 | 573.389 | −49.34 |
| TTM069 | −11.61 | −55.21 | −2.885 | −6.377 | 693.179 | −0.471 | 742.727 | −23.81 |
| TTM070 | −11.603 | −48.6 | −4.831 | −2.061 | 452.041 | 0.205 | 1050.915 | −29.6 |
| TTM071 | −11.599 | −66.86 | −4.242 | −1.989 | 401.526 | 0.251 | 748.67 | −25.88 |
| TTM072 | −11.586 | −60.37 | −5.246 | −4.661 | 1344.786 | −0.171 | 2964.319 | −42.15 |
| TTM073 | −11.584 | −72.1 | −2.174 | −4.218 | 646.718 | −0.533 | 803.018 | −44.03 |
| TTM074 | −11.582 | −47.9 | −4.053 | −4.884 | 6691.728 | 0.502 | 10000 | −49.15 |
| TTM075 | −11.573 | −43.51 | −5.48 | −4.347 | 356.286 | 0.336 | 792.851 | −47.45 |
| TTM076 | −11.569 | −53.72 | −4.832 | −4.53 | 492.296 | −0.952 | 541.067 | −44.59 |
| TTM077 | −11.561 | −63.12 | −5.28 | −3.991 | 520.147 | −0.944 | 641.872 | −37.65 |
| TTM078 | −11.544 | −47.48 | −4.343 | −4.668 | 979.919 | −0.185 | 3194.549 | −56.04 |
| TTM079 | −11.53 | −51.02 | −5.471 | −2.976 | 823.447 | 0.415 | 443.651 | −41.17 |
| TTM080 | −11.498 | −46.18 | −5.088 | −3.847 | 754.245 | 0.711 | 3160.81 | −51.52 |
| TTM081 | −11.496 | −56.42 | −4.888 | −2.912 | 350.398 | 0.008 | 1153.561 | −42.79 |
| TTM082 | −11.495 | −61.78 | −5.139 | −1.461 | 457.032 | 0.962 | 709.696 | −36.45 |

A—MMGBSA dG Bind
B—QPlogHERG
C—CIQPlogS
D—QPPCaco
E—QplogBB
F—QPPMDCK
G—ΔGtotal*

The identified compounds, their pharmaceutically acceptable salts (e.g., chlorides, acetates, formates, etc.), and their diastereomers show promise as improved drug candidates and leads for the treatment of neuropsychiatric diseases that benefit from psilocin treatment.

The rigor of the methodology used. The incorporation of a membrane in all simulations is becoming more common but is not the norm. Membrane protein analyses that do not consider the membrane are of little use. A lot of the previous work in this space has that problem.

We relied on the newly published crystal structures. These are only recently released, meaning this work has only been possible very briefly.

We identified new chemical material that has never been synthesized to the best of our knowledge. These are jumping off points for 2nd generation design once we make or obtain these molecules.

These are likely potent binders to the serotonin receptor. The downstream effects are difficult to predict without more data, but these molecules will help us generate that data, and may be drugs in their own right.

What is claimed is:

1. A therapeutically effective composition comprising [1-(1H-indol-3-yl)propan-2-yl][2-(oxolan-3-yl)ethyl]amine (TTA005); (1-cyclobutylpropan-2-yl)[2-(6-fluoro-1H-indol-3-yl)ethyl]amine (TTA009); [2-(6-fluoro-1H-indol-3-yl)ethyl](3-methylhexan-2-yl)amine (TTA011); [2-(6-fluoro-1H-indol-3-yl)ethyl](5-methylhexan-2-yl)amine (TTA012); [1-(5-fluoro-1H-indol-3-yl)propan-2-yl][(2R)-2-hydroxy-3-methoxypropyl]amine (TTA015); 1-{[4-(1H-indol-3-yl)-4-methylpentan-2-yl]amino}-3-methylbutan-2-ol (TTA0-19); 2-{[2-(5-fluoro-1H-indol-3-yl)ethyl]amino}propan-1-ol (TTA020); 2-{[1-(5-fluoro-1H-indol-3-yl)propan-2-yl]amino}-N-(prop-2-en-1-yl)propenamide (TTA024); 3-{[1-(5-fluoro-1H-indol-3-yl)propan-2-yl]amino}butan-2-ol (TTA031); (4-cyclopropylbutan-2-yl)[2-(6-fluoro-1H-indol-3-yl)ethyl]amine (TTA033); (4,4-dimethylhexan-3-yl)[2-(6-fluoro-1H-indol-3-yl)ethyl]amine (TTA035); (1-ethoxypropan-2-yl)[2-(6-fluoro-1H-indol-3-yl)ethyl]amine (TTA036); 2-{[1-(5-fluoro-1H-indol-3-yl)propan-2-yl]amino}-N-(prop-2-yn-1-yl)propenamide (TTA037); 3-{[2-(6-fluoro-1H-indol-3-yl)ethyl]amino}-2-methylbutan-2-ol (TTA038); [1-(1H-indol-3-yl)propan-2-yl][1-(thiophen-3-yl)ethyl]amine (TTA039), ligands TTM001, TTM002, TTM003, TTM004, TTM005, TTM006, TTM009, TTM013, TTM017, TTM019, TTM024, TTM028, TTM034, TTM038, TTM041, TTM045, TTM048, TTM057, TTM059, TTM068, TTM082, their pharmaceutically acceptable salts, or diastereomers thereof.

2. A therapeutically effective composition according to claim 1 comprising: [1-(1H-indol-3-yl)propan-2-yl][1-(thiophen-3-yl)ethyl]amine (TTA039); (1-cyclobutylpropan-2-yl)[2-(6-fluoro-1H-indol-3-yl)ethyl]amine (TTA009); [1-(5-fluoro-1H-indol-3-yl)propan-2-yl][(2R)-2-hydroxy-3-methoxypropyl]amine (TTA015); 2-{[1-(5-fluoro-1H-indol-3-yl)propan-2-yl]amino}-N-(prop-2-yn-1-yl)propenamide (TTA037); (4,4-dimethylhexan-3-yl)[2-(6-fluoro-1H-indol-3-yl)ethyl]amine (TTA035); 3-{[2-(6-fluoro-1H-indol-3-yl)ethyl]amino}-2-methylbutan-2-ol (TTA038); (4-cyclopropylbutan-2-yl)[2-(6-fluoro-1H-indol-3-yl)ethyl]amine (TTA033); [2-(6-fluoro-1H-indol-3-yl)ethyl](5-methylhexan-2-yl)amine (TTA012); [2-(6-fluoro-1H-indol-3-yl)ethyl](3-methylhexan-2-yl)amine (TTA011); or [1-(1H-indol-3-yl)propan-2-yl][2-(oxolan-3-yl)ethyl]amine (TTA005).

3. A therapeutically effective composition according to claim 1 comprising: [1-(1H-indol-3-yl)propan-2-yl][1-(thiophen-3-yl)ethyl]amine (TTA039); (1-cyclobutylpropan-2-yl)[2-(6-fluoro-1H-indol-3-yl)ethyl]amine (TTA009); [1-(5-fluoro-1H-indol-3-yl)propan-2-yl][(2R)-2-hydroxy-3-methoxypropyl]amine (TTA015); 2-{[1-(5-fluoro-1H-indol-3-yl)propan-2-yl]amino}-N-(prop-2-yn-1-yl)propenamide (TTA037); or (4,4-dimethylhexan-3-yl)[2-(6-fluoro-1H-indol-3-yl)ethyl]amine (TTA035).

4. A therapeutic treatment process that comprises administering to a human or mammal subject in need thereof a therapeutically-effective amount of: (a) an active ingredient comprising [1-(1H-indol-3-yl)propan-2-yl][2-(oxolan-3-yl)ethyl]amine (TTA005); (1-cyclobutylpropan-2-yl)[2-(6-fluoro-1H-indol-3-yl)ethyl]amine (TTA009); [2-(6-fluoro-1H-indol-3-yl)ethyl](3-methylhexan-2-yl)amine (TTA011); [2-(6-fluoro-1H-indol-3-yl)ethyl](5-methylhexan-2-yl)amine (TTA012); [1-(5-fluoro-1H-indol-3-yl)propan-2-yl][(2R)-2-hydroxy-3-methoxypropyl]amine (TTA015); 1-{[4-(1H-indol-3-yl)-4-methylpentan-2-yl]amino}-3-methylbutan-2-ol (TTA0-19); 2-{[2-(5-fluoro-1H-indol-3-yl)ethyl]amino}propan-1-ol (TTA020); 2-{[1-(5-fluoro-1H-indol-3-yl)propan-2-yl]amino}-N-(prop-2-en-1-yl)propenamide (TTA024); 3-{[1-(5-fluoro-1H-indol-3-yl)propan-2-yl]amino}butan-2-ol (TTA031); (4-cyclopropylbutan-2-yl)[2-(6-fluoro-1H-indol-3-yl)ethyl]amine (TTA033); (4,4-dimethylhexan-3-yl)[2-(6-fluoro-1H-indol-3-yl)ethyl]amine (TTA035); (1-ethoxypropan-2-yl)[2-(6-fluoro-1H-indol-3-yl)ethyl]amine (TTA036); 2-{[1-(5-fluoro-1H-indol-3-yl)propan-2-yl]amino}-N-(prop-2-yn-1-yl)propenamide (TTA037); 3-{[2-(6-fluoro-1H-indol-3-yl)ethyl]amino}-2-methylbutan-2-ol (TTA038); [1-(1H-indol-3-yl)propan-2-yl][1-(thiophen-3-yl)ethyl]amine (TTA039), their pharmaceutically acceptable salts, or diastereomers thereof with (b) one or more pharmaceutically acceptable carriers or excipients.

5. A process according to claim 4 wherein the administered active ingredient comprises [1-(1H-indol-3-yl)propan-2-yl][1-(thiophen-3-yl)ethyl]amine (TTA039); (1-cyclobutylpropan-2-yl)[2-(6-fluoro-1H-indol-3-yl)ethyl]amine (TTA009); [1-(5-fluoro-1H-indol-3-yl)propan-2-yl][(2R)-2-hydroxy-3-methoxypropyl]amine (TTA015); 2-{[1-(5-fluoro-1H-indol-3-yl)propan-2-yl]amino}-N-(prop-2-yn-1-yl)propenamide (TTA037); (4,4-dimethylhexan-3-yl)[2-(6-fluoro-1H-indol-3-yl)ethyl]amine (TTA035); 3-{[2-(6-fluoro-1H-indol-3-yl)ethyl]amino}-2-methylbutan-2-ol (TTA038); (4-cyclopropylbutan-2-yl)[2-(6-fluoro-1H-indol-3-yl)ethyl]amine (TTA033); [2-(6-fluoro-1H-indol-3-yl)ethyl](5-methylhexan-2-yl)amine (TTA012); [2-(6-fluoro-1H-indol-3-yl)ethyl](3-methylhexan-2-yl)amine (TTA011); or [1-(1H-indol-3-yl)propan-2-yl][2-(oxolan-3-yl)ethyl]amine (TTA005).

6. A process according to claim 5 wherein the administered active ingredient comprises [1-(1H-indol-3-yl)propan-2-yl][1-(thiophen-3-yl)ethyl]amine (TTA039); (1-cyclobutylpropan-2-yl)[2-(6-fluoro-1H-indol-3-yl)ethyl]amine (TTA009); [1-(5-fluoro-1H-indol-3-yl)propan-2-yl][(2R)-2-hydroxy-3-methoxypropyl]amine (TTA015); 2-{[1-(5-fluoro-1H-indol-3-yl)propan-2-yl]amino}-N-(prop-2-yn-1-yl)propenamide (TTA037); or (4,4-dimethylhexan-3-yl)[2-(6-fluoro-1H-indol-3-yl)ethyl]amine (TTA035).

7. A method for the treatment of depression, comprising administering an effective amount of a composition comprising a selective 5-HT2A inverse agonist to a human in need of such treatment, wherein the compound is selected from [1-(1H-indol-3-yl)propan-2-yl][2-(oxolan-3-yl)ethyl]amine (TTA005); (1-cyclobutylpropan-2-yl)[2-(6-fluoro-1H-indol-3-yl)ethyl]amine (TTA009); [2-(6-fluoro-1H-indol-3-yl)ethyl](3-methylhexan-2-yl)amine (TTA011); [2-(6-fluoro-1H-indol-3-yl)ethyl](5-methylhexan-2-yl)amine (TTA012); [1-(5-fluoro-1H-indol-3-yl)propan-2-yl][(2R)-2-hydroxy-3-methoxypropyl]amine (TTA015); 1-{[4-(1H-indol-3-yl)-4-methylpentan-2-yl]amino}-3-methylbutan-2-ol (TTA0-19); 2-{[2-(5-fluoro-1H-indol-3-yl)ethyl]amino}propan-1-ol (TTA020); 2-{[1-(5-fluoro-1H-indol-3-yl)propan-2-yl]amino}-N-(prop-2-en-1-yl)propenamide (TTA024); 3-{[1-(5-fluoro-1H-indol-3-yl)propan-2-yl]amino}butan-2-ol (TTA031); (4-cyclopropylbutan-2-yl)[2-(6-fluoro-1H-indol-3-yl)ethyl]amine (TTA033); (4,4-dimethylhexan-3-yl)[2-(6-fluoro-1H-indol-3-yl)ethyl]amine (TTA035); (1-ethoxypropan-2-yl)[2-(6-fluoro-1H-indol-3-yl)ethyl]amine (TTA036); 2-{[1-(5-fluoro-1H-indol-3-yl)propan-2-yl]amino}-N-(prop-2-yn-1-yl)propenamide (TTA037); 3-{[2-(6-fluoro-1H-indol-3-yl)ethyl]amino}-2-methylbutan-2-ol (TTA038); [1-(1H-indol-3-yl)propan-2-yl][1-(thiophen-3-yl)ethyl]amine (TTA039), ligands TTM001, TTM002, TTM003, TTM004, TTM005, TTM006, TTM009, TTM013, TTM017, TTM019, TTM024, TTM028, TTM034, TTM038, TTM041, TTM045, TTM048, TTM057, TTM059, TTM068, TTM082, and their pharmaceutically acceptable salts, or diastereomers thereof.

8. A method according to claim 7 wherein said composition is formulated for oral, sublingual, intranasal, pulmonary administration, buccal, sublingual, rectal, transdermal, transmucosal, epidural, intrathecal, intraocular topical, creams, lotions, gels, and eye drops.
